(19) United States
(12) Patent Application Publication
(10) Pub. No.: US 2023/0033676 A1
(43) Pub. Date: Feb. 2, 2023

(54) ROBOTIC DILATOR

(71) Applicant: ESSENTIAL MACHINES, INC., Santa Clara, CA (US)

(72) Inventors: Alexander Todorov, Santa Clara, CA (US); Xingchi He, Cupertino, CA (US)

(21) Appl. No.: 17/514,985

(22) Filed: Oct. 29, 2021

Related U.S. Application Data
(60) Provisional application No. 63/226,992, filed on Jul. 29, 2021.

(51) Int. Cl. A61B 17/02; A61B 34/30

(52) U.S. Cl. CPC A61B 17/0206; A61B 17/0218; A61B 34/30; A61B 2017/0262; A61B 2090/064

(57) ABSTRACT

A dilator tool for use with a robotic surgical system is disclosed. In some embodiments, the dilator comprises a set of elongate members with an atraumatic form. The elongate members are individually controlled to manipulate tissue and push tissue away from a working channel. The operation to create a working channel may reduce the risk of tissue injury over traditional methods using a scalpel.

### CROSS-REFERENCE TO RELATED APPLICATIONS

This application claims priority in part from U.S. Provisional Application 63/226,992, entitled “Robotic Dilator” and filed on Jul. 29, 2021, the contents of which are herein incorporated by reference in its entirety.

### BACKGROUND

Tubular dilators as known in the art are generally inserted in telescopic fashion until the necessary surgical channel is created. There are also manual distractors which consist of manually adjustable number of blades for establishing the surgical channel. Both of these approaches are manual and do not provide the surgeon with any meaningful feedback other than retracting the tissue.

### SUMMARY

Described herein are various embodiments for a tissue dilator device, as well as systems and methods for using the same.

The robotic dilator described herein, is a multi-blade (e.g., two or more blades) support structure used to establish and maintain a surgical channel. The surgical channel may allow a surgeon, or a surgical robot, to have access to a point of interest on the spine. An electronic controller provides the capability to individually actuate/control each individual blade of the two or more blades, which are used to establish and form of a surgical channel. The dilator blades form a narrow diameter tube in one position, and while in the narrow diameter tube, the dilator blades can be inserted into the patient. The narrow diameter tube forms the minimum diameter/footprint of the robotic dilator. Once the robotic dilator is in place, the individual blades may then be actuated individually (one at a time) or simultaneously (2 or more at a time) to dilate the surrounding tissue. The surgical channel may be expanded using the blades, until the channel is large enough for surgical tools to pass through the surgical channel created by the dilator blades. Each dilator blade may be equipped with one or more force sensors, to measure any resistance force the tissue may apply to the blades as the working channel is created or maintained. The information from the force sensors may help guide the surgeon to avoid inducing unnecessary trauma to a patient’s surgical area. In some examples, one or more neurostimulator sensors may be implemented. Each dilator blade may also be equipped with neuromonitoring electrodes, for determining the proximity of each individual blade to a potential surrounding nerve route, thus providing the surgeon with information helping to avoid nerve damage or trauma.

In an embodiment there may be a tissue dilating apparatus for use with a robotic surgery system.

In an embodiment, there is a blade assembly apparatus. The blade assembly apparatus has a frame housing. The frame housing has a front plate and a back plate, and two or more support members between the front plate and the back plate. There is also a first screw mechanically engaged to the housing. The first screw passing through the back plate and is coupled to the front plate. The coupling may be facilitated mechanically. The first screw has a front end, a back end, and a first screw longitudinal axis. The first screw in some examples has a first gear along a first screw length, the first gear is near the back plate. The blade assembly apparatus also has a carriage mechanically engaged to the first screw, such that rotation of the first screw can displace the carriage along the first screw longitudinal axis of the first screw, or in some examples, at least a portion of a length of the first screw. There is also a blade attached or mechanically engaged to the carriage, the blade is positioned outside the housing. The blade assembly apparatus allows for rotation of the first screw, to cause the carriage to move within the housing, and the displacement of the blade outside of the housing. The first screw may include a first translation gear.

In some embodiments, the blade assembly may have additional components which may or may not be used. The design may be altered to fit a particular clinical need. The blade assembly may have a second screw generally parallel to the first screw. The second screw may also be supported between the back plate and the front plate. The second screw may have a second gear that is positioned along the length of the second screw so as to generally match the position of the gear on the first screw. The second screw may have a second translation gear in substantially orthogonal alignment with the first translation gear of the first screw. The second screw may have a second screw longitudinal axis. The gears (e.g., the first screw, the second screw, etc.) may mesh and one gear drive the other, or there may be a pinion gear between them. In the case where the gears directly drive each other, the first screw and the second screw may rotate in opposite directions. The pinion gear may be in rotational engagement with the first translation gear and the second translation gear such that the pinion gear is configured to provide force to drive the second translation gear when the first translation gear is rotated. A rotation of the first and second translation gears is configured to move the carriage along the first screw longitudinal axis and the second screw longitudinal axis. Where a pinion gear is used, the first and second screw may rotate in the same direction. In some embodiments, there may be a transverse screw to cause the carriage to move in a transverse direction relative to the first screw.

In some embodiments, there is a tissue dilating device for use with a robotic surgical system. In certain examples, the tissue dilating apparatus has a frame having an interior and an exterior. Two or more blade assemblies are mounted circumferentially around the exterior of the frame, each blade assembly has a housing, a first screw mechanically engaged to the housing, a carriage mechanically engaged to the first screw, such that rotation of the first screw can displace the carriage along at least a portion of a length of the first screw and a blade fixedly attached to the carriage, the blade positioned outside the housing, wherein the rotation of the first screw causes the carriage to move within the housing, and causes the displacement of the blade outside of the housing and in the interior of the frame. In some examples, the blade is made of at least one selected from a group consisting of a stainless steel, an aluminum, a titanium and a metal alloy. The tissue dilating apparatus may also have two or more mechanical actuators engaged to a like number of blade assemblies. Each of the mechanical actuators may be mechanically engaged or coupled to a respective one of the plurality of blade assemblies. In this way, the plurality of mechanical actuators move the plurality of blade assembly apparatus to create a working channel for a surgical instrument.

In some embodiments, when the elongate members are in a closed position, the elongate members may be spaced equidistant around the aperture created by the interior surface of the frame. Each elongate member may be moved in a radial direction moving in and out from the center. When the elongate members are pushed toward the center of the aperture and abut each other along their elongate length, they form a closed position, and the elongate members may not be “closed” any further. Each elongate member has a closed position, and a variety of open positions. There may be two, three or four elongate blades in some embodiments. There may be more than four elongate blades in some other embodiments. In some embodiments, the elongate members may come together when the elongate members are closed in around the axis of operation, and form a narrow working channel. In other embodiments, the elongate members may be moved away from the axis of operation, and moved toward the edges of the aperture defined by the inner surface of the frame. In some embodiments, when the elongate members are moved away from the axis of operation the elongate members may be moved in an irregular pattern or manner from each other. The elongate members may form any combination of positions as may be allowed using the motors and gears to power the elongate members, or any other sub component described herein.

In various embodiments, the elongate members may have a proximal end and a distal end. For purposes of discussion and orientation, the proximal end refers to the end of something that is closest to the user, while the distal end refers to the end farthest from the user, or closest to the surgical site. In various embodiments, the elongate members may not be connected to each other along their length, but may instead operate independently of each other.

In some embodiments, there may be a tissue dilating apparatus according to the previous aspect, wherein the plurality of elongate members are not connected to each other.

In some embodiments, there may be a tissue dilating apparatus wherein the plurality of elongate members further comprises a first elongate member, and a second elongate member. The first and second elongate members may be moveably attached to the frame. The first and second elongate members each have a proximal end in close proximity to the frame. The first and second elongate members each have a distal end. An expandable barrier may be mechanically engaged to the distal end of the first and second elongate members.

In some embodiments, there may be a tissue dilating apparatus wherein one or more of the elongate members further comprises a carriage, the carriage bridging the distance of the frame. The carriage may be in mechanical engagement with at least one of the motor gears.

In some embodiments, the tissue dilating apparatus may have elongate members that may be movable about a fulcrum.

In some embodiments, the tissue dilating apparatus may have a fulcrum positioned in the aperture defined by the interior surface of the frame.

In some embodiments, the tissue dilating apparatus may have a plurality of elongate members wherein each of the elongate members may be independently movable.

In some embodiments, the tissue dilating apparatus may have a frame that is movable about the axis of operation.

In some embodiments, the tissue dilating apparatus may also have a plurality of motors in mechanical engagement with the set of motor gears.

In some embodiments, the tissue dilating apparatus may include an electronic controller for controlling the plurality of motors.

In some embodiments, there may be a tissue dilating apparatus as described herein that may include a force sensor operable with at least one of the elongate members.

In some embodiments, there may be a tissue dilating apparatus as described herein that further comprises at least one neuromonitoring sensor in mechanical engagement with at least one of the elongate members.

In some embodiments, there may be a tissue dilating apparatus as described herein wherein the frame may define a plane that may be substantially orthogonal to the axis of operation.

In some embodiments, there may be a tissue dilating apparatus as described herein, wherein the plurality of elongate members may be operable as a tissue retractor.

In some embodiments, there may be a robotic surgery system with a tissue dilating device. The system possesses a robotic arm and a tissue dilating apparatus positioned at the distal end of the robotic arm. The apparatus has a frame with an interior surface and an exterior surface. The interior surface of the frame defines an aperture. A plurality of elongate members with an atraumatic form are disposed in a generally orthogonal orientation with respect to the aperture. The plurality of elongate members define a working channel, and an axis of operation. A plurality of gears are in moveable mechanical engagement with the plurality of elongate members. The gears are mechanically engaged to the exterior surface of the frame. The plurality of gears move in response to a plurality of motors, the gears moving the elongate members to alter the shape of the working channel. A computer with a user interface is in electrical communication with robotic arm and the tissue dilating apparatus. There is an electronic controller directing the movement of the robotic arm and the tissue dilating apparatus, the electronic controller is operated through the user interface of the computer.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a tissue dilating device in accordance with an embodiment.

FIG. 2 illustrates a top view of a tissue dilating device in accordance with an embodiment.

FIGS. 3A-B illustrate two additional top views of a tissue dilating device in accordance with some embodiments.

FIG. 4 illustrates a blade gear assembly of a tissue dilating device in accordance with an embodiment.

FIGS. 5A-B illustrate a blade assembly of a tissue dilating device in accordance with several embodiments.

FIG. 6 illustrates a cross section view of a portion of a blade assembly in accordance with an embodiment.

FIG. 7 illustrates two positions of the blade assembly in accordance with an embodiment.

FIG. 8 illustrates a blade assembly mounted to a frame in accordance with an embodiment.

FIG. 9 illustrates a second position of a blade assembly mounted to a frame in accordance with an embodiment.

FIG. 10 illustrates a tapered dilator in accordance with an embodiment.

FIG. 11 illustrates two different blade positions in accordance with some embodiments.

FIG. 12 illustrates several possible blade cross sections in accordance with several embodiments.

FIG. 13 illustrates an example blade orientation in accordance with an embodiment.

FIG. 14 illustrates several distal end positions of elongate members in accordance with some embodiments.

FIG. 15 illustrates a tissue dilator housing and a motor controller housing in accordance with an embodiment.

FIG. 16 illustrates a side view of a tissue dilator housing and a motor controller housing in accordance with an embodiment.

FIG. 17 illustrates an example of user control and guidance of a tissue dilator according to an embodiment.

FIGS. 18A-G illustrate an example method of using a tissue dilator in accordance with an embodiment.

FIGS. 19A-B illustrate an alternate example method of using a tissue dilator in accordance with an embodiment.

FIGS. 19C-D illustrate an alternate example method of using a tissue dilator in accordance with an embodiment.

FIG. 20 illustrates methods of controlling a tissue dilator in accordance with an embodiment.

FIG. 21 illustrates example elongate member positions in response to a control method in accordance with an embodiment.

FIGS. 22A-22D illustrates various embodiments of closed elongate member forms according to several embodiments.

FIGS. 23A-23D illustrates various examples of elongate member cross section views in accordance with several embodiments.

FIGS. 24A-24D illustrate various embodiments of working channels formed from elongate members in closed positions in accordance with several embodiments.

FIGS. 25A-25D illustrate various embodiments of working channels formed from elongate members in open positions in accordance with several embodiments.

FIGS. 26A and 26B illustrate an open and closed arrangement respectively of elongate members in accordance with an embodiment.

FIG. 27 illustrates an example working channel defined by differently positioned elongate members in accordance with an embodiment.

FIG. 28 illustrates an example of a dilator assembly according to an embodiment.

FIG. 29 is a simplified flow diagram showing a method for using a dilator according to certain embodiments of the present disclosure.

### DETAILED DESCRIPTION

In the various figure descriptions, part numbers are assigned to drawing elements without regard to importance. A lower part number does not signify greater or lesser importance. Part numbers are arbitrarily assigned during the drafting the patent application. No commonality exists or is intended between similar parts of different drawings.

As used herein, when an element, component, device or layer is described as being “on,” “connected to,” “coupled to” or “in contact with” another element, component, device or layer, it can be directly on, directly connected to, directly coupled with, in direct contact with, or intervening elements, components, devices or layers may be on, connected, coupled or in contact with the particular element, component or layer, for example. When an element, component, device or layer for example is referred to as being “directly on,” “directly connected to,” “directly coupled to,” or “directly in contact with” another element, component, device or layer, there are no intervening elements, components, devices or layers for example.

Described herein are various embodiments of a medical tool for use with a robotic surgery device or system. For discussion purposes only, a frame of reference is adopted to assist in the reading of the present disclosure. Reference is made to a surgical site, which is the location in a patient body where a medical procedure may occur. The physical elements of the various embodiments describe any portion of the element that is closest to the surgical site position as distal or distal end. Elements which have a portion that is toward the surgical site have a distal portion. If the element also has a portion that is further away from the surgical site, that further away portion is referred to as the proximal end.

In some embodiments, the use of “front” and “back” are used. The front is closest to generally facing toward the surgical site, or facing the working channel that connects to the surgical site. The back end is away from the surgical site, or the side opposite the front end.

To avoid any prolix description of the many embodiments, the use of “generally” or “substantially” are used herein and meant to refer to the written description of an object, plus or minus, an additional 15% of the particular measurement. If the reference is in degrees, such as an object that is “substantially orthogonal”, the relationship may be read as 90 degrees, +/- 15 degrees. If the measurement is “substantially 100 millimeters”, the measurement should be read as 100 mm, +/- 15 mm (15%).

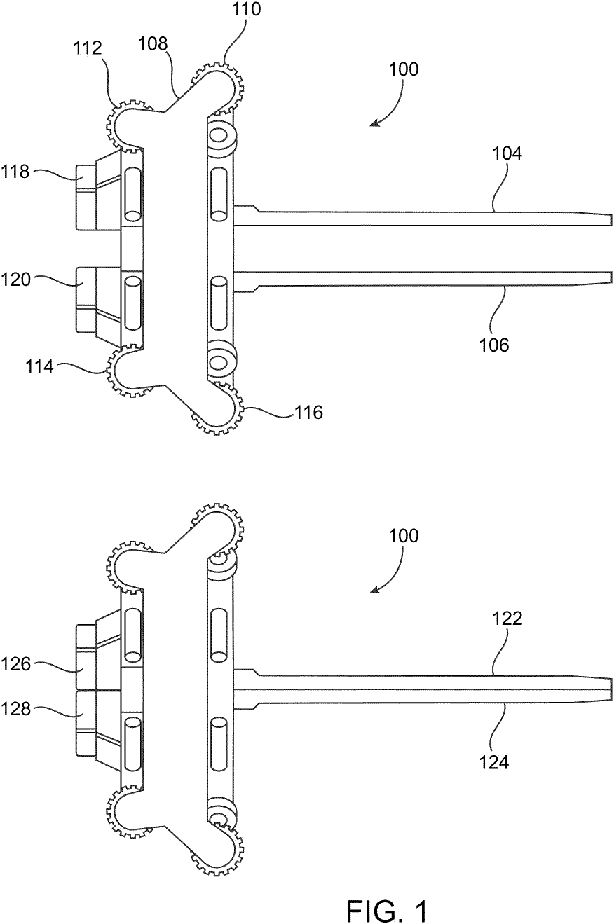

FIG. 1 illustrates a side view of a tissue dilating device 100. In some embodiments, the tissue dilating device 100 may have a set of elongate member assemblies (also referred to herein as blade assemblies, and used interchangeably). The blade assemblies 118, 120 may be mounted to a frame 108 in such a way that the blades 104, 106, also referred to as elongate members, extend through an opening of the frame. The blade assemblies 118, 120 may be moved, causing the blades 104, 106 to open or close. In an embodiment, there may be four blade assemblies mounted around the perimeter of the frame 108. Each blade assembly may be driven by a different gear 110, 112, 114, 116. The side view of FIG. 1 shows two blade assemblies 118, 120, with two hidden behind the two shown. In an embodiment, the blades 104, 106 may be seen in an open position, with a gap between the two blades.

In another embodiment, the two blades 122, 124 may be in a closed position, with no gap viewable between the blades 122, 124. The blade assemblies 126, 128 may be seen to abut each other, with no gap between them.

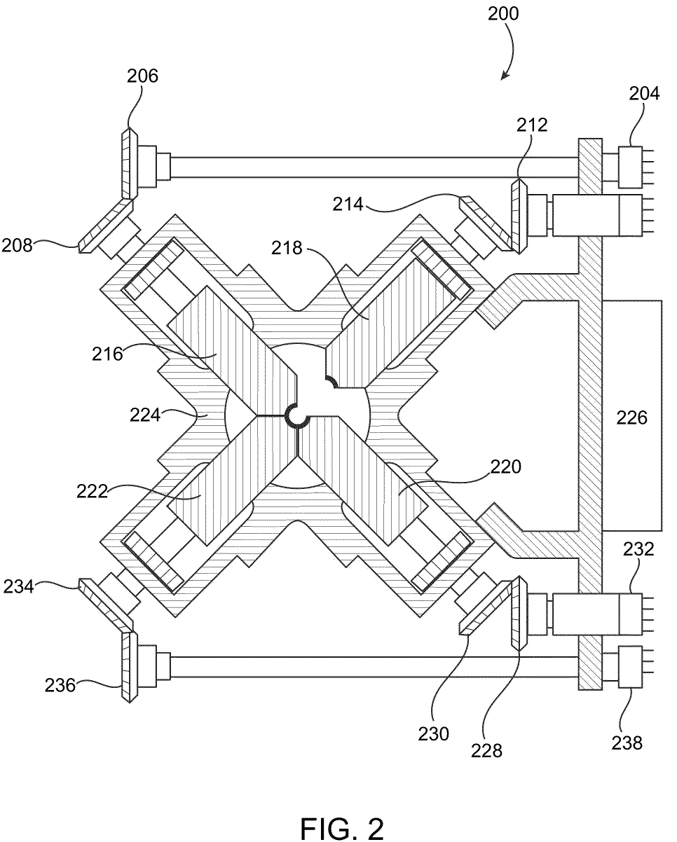

A top view of the frame 224, caps, and motor gear assembly 200 are shown in FIG. 2. In an embodiment, the motor gear assembly 200 may include a set of motor gears that engage a set of blade assemblies 216, 218, 220, 222. The blade assemblies may be fastened or attached to the frame 224. In an embodiment, a first motor gear 204 may rotate and drive a first translation gear 206, where the first motor gear and the first translation gear may be on the same axle. The first translation gear 206 may be mechanically engaged with a first blade gear 208. The first blade gear 208 may be part of, or mechanically engaged with a lead screw that may move the carriage and blade in an axial direction that may be the same as the axis of the drive screw.

In some embodiments, each blade assembly may have a separate translation gear. In an embodiment, a second translation gear 212 may engage a blade gear 214 to move a second blade 218. In an embodiment, a third motor gear 232 may drive a third translation gear 228, which in turn may drive a third blade gear 230 to move a third blade 220. A fourth motor gear 238 may drive a fourth translation gear 236, which may mechanically engage a fourth blade gear 234 to move a fourth blade 222.

The frame 224 may have an electrical connector 226 for a general purpose input/output device. This may be a quick connect electrical socket having multiple connectors for different components such as power, force sensing elements, neuromonitoring signals, stress and/or strain sensors, position sensors and so on. The various electrical components may be wired into a single adapter, or may maintain their separate identity with a variety of plugs in the electrical connector 226.

In some embodiments, the frame may be rotated, so the blade assemblies may rotate in a circular fashion. The rotation of the frame about the axis of operation may be conducted manually, or under motor control. The motor control may also function as a fully robotic motion, or a robot assisted motion. In the various embodiments, the frame may rotate through an arc measure that matches half the angle between the elongate members. For example, if there are three elongate members, the arc measure between the three elongate members may be 120 degrees. The frame may rotate ½ of the arc measure, or rotate up to 60 degrees. If there are four elongate members, the arc measure may be 90 degrees between the elongate members. In this example the frame may rotate about 45 degrees, or half the arc measure between the elongate members. In still other embodiments, the frame may rotate at least ½ the arc measure between the elongate members. In other embodiments, the frame may rotate freely.

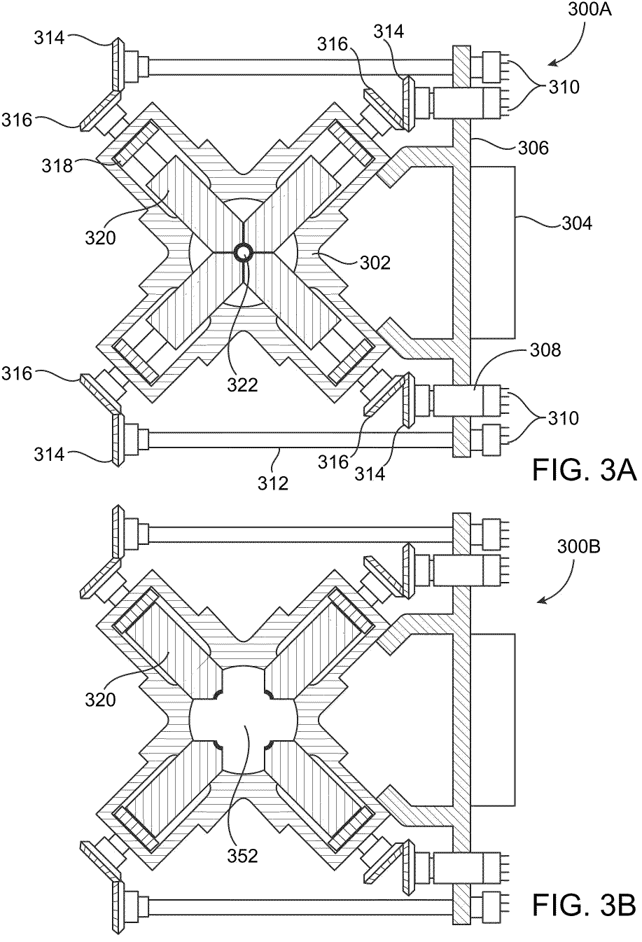

A view of two embodiments of the dilator are now seen in FIG. 3A and FIG. 3B. The dilator may be seen with a symmetric closed position in FIG. 3A with a frame which may be used to support four different blade assemblies as seen from a top view. The cap portion of each blade may be seen in a closed position, with a small aperture 322 formed from the curved face of the four cap portions as shown. The aperture may be curved and formed in a circular form in this embodiment. The aperture may have any desired shape. In some embodiments, each blade cap may make a portion of the aperture shape, with the portion of the aperture perimeter divided among the number of blade assemblies. As described herein, there may be fewer or more blade assemblies, which may change the shape of the center aperture. For example there may be five blade assemblies which may form a pentagon shaped aperture. There may be six blade assemblies that may form a hexagon shaped aperture, and so on. In some embodiments, the shape of the aperture may not reflect the number of blade assemblies. For example, the center aperture of the closed blade assemblies may be a circle, but there may be only 2 blade assemblies. In another example embodiment, there may be five blade assemblies, and the aperture formed by the blade caps when in a closed configuration may be a triangle. The shape of the aperture may be any desired, and formed into the caps and/or blades of the blade assemblies.

In an embodiment, the other features shown in FIG. 3A are the dilator top view 300A with the blade assemblies in a closed position. A frame 302 may support the various elements of the dilator 300A. The frame 302 may be a single contiguous part, or structure made of several components. The frame 302 may have a connector bar support 306 mechanically engaged to the frame 302. The connector bar support 306 may support or carry a connector 304 which may be a connector of any type as described herein. The connector bar support may support or mechanically engage a set of motor gears 310. The set of motor gears may receive rotational energy or force, from a set of motors. The set of motor gears 310 may translate force through a set of drive axles, two which may be short and two which may be longer. Each of the drive axles may terminate in a translation gear 314 which may translate force to a set of blade gears 316. The blade assemblies 320 include a carriage and a blade, as well as a set of blade gears as described herein. The predominant component viewable from the top in each blade assembly is the cap of each blade. The cap may be part of the blade, the carriage or an independent component, depending on the design of the blade assembly. The blade assemblies may form an aperture 322 when the blade assemblies 320 are in a closed position as shown in FIG. 3A. The aperture 322 may be sized to fit a guide wire, an introducer, or other slender tool through the aperture. The tool may be inserted through the aperture from the top, from the bottom, or the aperture may be closed around the tool (the tool may be placed into the frame opening when the blade assemblies are open, and the blade assemblies may be closed onto the tool).

In another view, the blade assemblies 320 may be seen in an open configuration as shown in FIG. 3B. In an embodiment, the dilator in the open position 300B may have the same components as in the closed position, however the individual blade assemblies may be viewed as being moved on the axles of the blade gears away from the center, and creating a larger aperture 352 in the center of the frame 302. The open aperture 352 and frame 302 may be selected of varying sizes to facilitate different types of surgical procedures, as well as permitting one or more surgical tools to be inserted through the open aperture 352 during a medical procedure.

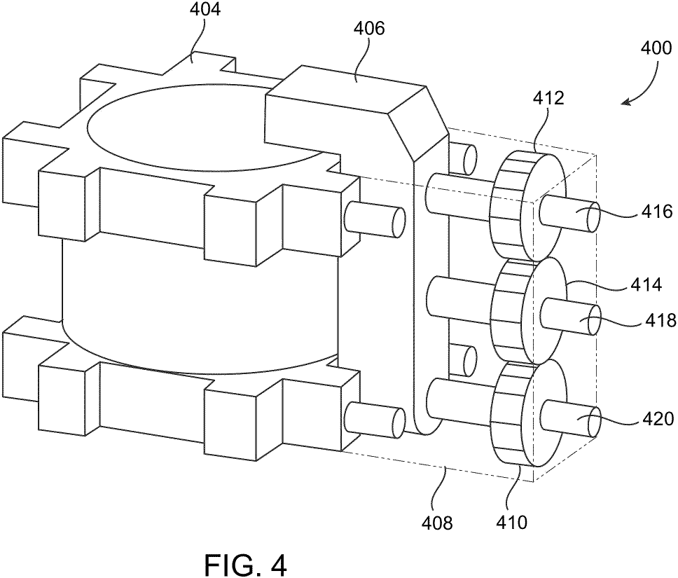

A blade gear assembly 400 is now shown in FIG. 4. In an embodiment, the frame 404 may have an adapter for each blade assembly that may be fitted to the frame. In an embodiment, the frame 404 may have four adapter positions for four blade assemblies. A partial blade assembly is shown in one of the adapter positions. A carriage 406 may have an inverted “L” shape, serving as both carriage and as a cap, and connect with the blade (not shown). The carriage may be connected to a blade assembly mount 408. In an embodiment, the blade assembly mount 408 may contain a drive gear 410, on a lower lead screw 420. A pinion gear 414 on a pinion axle 418 and an upper drive gear 412 on an upper drive screw 416. In other embodiments, there may be a drive mechanism engaged with the upper drive screw 416. In some embodiments, there may be a center drive screw on the pinion axle 418, which may drive the upper gear and lower gear. In some embodiments there may be an upper and lower drive gear, to provide direct mechanical force to the upper gear and the lower gear, in which case the pinion gear and axle may be omitted.

In an embodiment, motive force from a motor or motor gear connector may drive the drive gear 410. Torque, or force may be applied to the pinion gear 414 and to the upper drive gear 412. The force from the motor or motor gear connector drives the lower drive screw 420 and the upper drive screw 416 simultaneously. The carriage may move along the axis of the upper and lower drive screw, causing the blade to move in one axis. As may be appreciated by those skilled in the art, any one of the axles shown may be the drive gear, with torque being transferred to any of the other gears and axles.

In some embodiments, there may be a blade assembly apparatus. The blade assembly apparatus may have a carriage housing. In some embodiments, the carriage housing has a front plate and a back plate. There may be two or more support members connecting the front plate to the back plate. In some embodiments, the support members may connect the corners or edges of the front and back plate, forming a cube or other three dimensional frame like structure. A first screw may be coupled to the carriage housing, with the first screw passing through the back plate and coupled to the front plate. In some embodiments, the first screw may be received by an aperture in the front plate. In some embodiments, the front plate may have a port attached to the front plate, wherein the port may receive the tip or front end of the first screw. The first screw may be directly coupled to the front plate either in direct contact with the front plate, or coupled to the front plate through one or more intermediate elements. In an embodiment, the tip or front end of the first screw may be axially secured by the connection to the front plate to reduce wobbling or lateral oscillation. The first screw may have a drive gear positioned outside the carriage housing, and a first gear positioned adjacent the back plate, either inside or outside the back plate. A carriage may be mechanically engaged with the first screw, which may be a lead screw, ball screw or similar device. The first screw may rotate and drive the carriage forward or backward along the length of the screw. In some embodiments, a blade may be attached to, or part of the carriage. The blade may be positioned outside the carriage housing. A cap or bridge component may connect the blade and the carriage. The cap or bridge may provide sufficient space between the carriage and the blade so the carriage housing may be attached to a frame of a dilating device. The gap space may be sufficient to permit the carriage to be moved along the length of the first screw, and have the blade move a corresponding distance, with the blade inside the frame of a dilating device.

In some embodiments, there may be a second screw generally parallel to the first screw. The second screw may have the same configuration with respect to the front plate and the back plate, with a translation gear on the second screw. In some embodiments, the second screw may be received by an aperture in the front plate. In some embodiments, the front plate may have a port attached to the front plate, wherein the port may receive the tip or front end of the first screw. The first screw may be directly coupled to the front plate either in direct contact with the front plate, or coupled to the front plate through one or more intermediate elements. In an embodiment, the tip or front end of the first screw may be axially secured by the connection to the front plate to reduce wobbling or lateral oscillation. The translation gear of the second screw may be on the same position as the translation gear on the first screw, so the translation gears may directly or indirectly mesh together. In an embodiment where an indirect mesh of gears may be used, a pinion gear may be positioned between the first translation gear and the second translation gear. Then the first screw rotates, the first translation gear may rotate as well, and the pinion gear may transfer mechanical energy to the second translation gear, and cause the carriage to move in response to the rotation of the two screws.

The first screw, second screw and transverse screw discussed may be a standard screw, with each receiving part being threaded. In some embodiments, the screw may be a lead screw or a ball screw or similar component suited for causing the carriage to move along the length of the screw while the screw rotates. Other components that may be functionally equal to a lead screw may also be used.

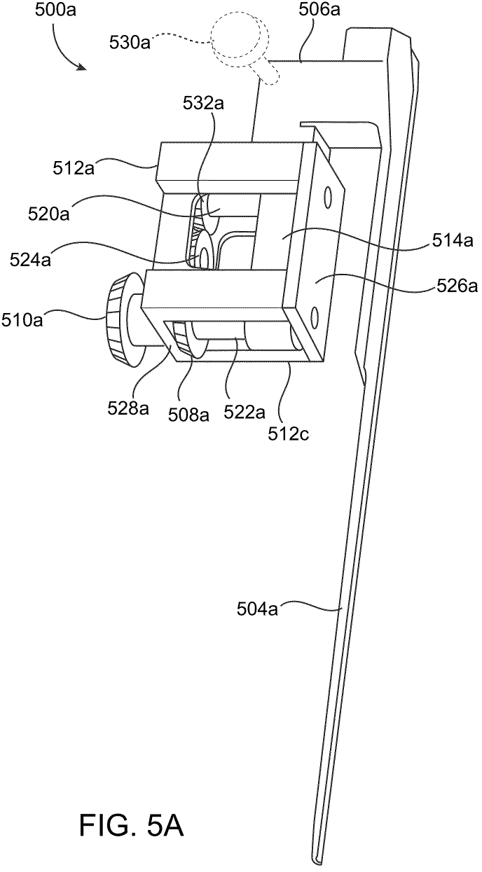

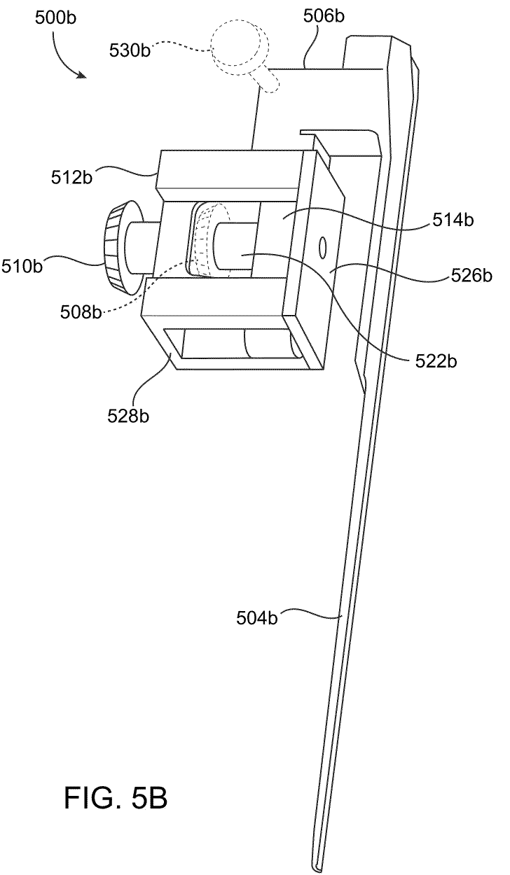

A blade assembly 500a is now shown in FIG. 5A. The blade assembly 500a includes an elongate member, such as a blade 504a. The blade 504a may have a cap 506a, and a carriage 514a. The carriage may have a first receptacle for receiving a first screw 522a, and a second receptacle for receiving a second screw 520a. The first screw 522a may be generally parallel to the support members of the carriage frame or housing 512a, and generally orthogonal to the front plate 526a and the back plate 528a. The first screw 522a may have a drive gear 510a and a translation gear 508a. The second screw 520a may have a second translation gear 532a. The first and second translation gears may be connected via a pinion gear 524a in rotational engagement between the first and second translation gears. The drive gear 510a may be driven by a motor, connector gear (to a motor) or a crank, causing force to be transferred to the second translation gear via the pinion gear.

In an embodiment, the carriage may move along the path defined by the first and second screws. The carriage 514a may move on the first and second screws with both screws turning in unison, or the movement of the carriage 514a on the second screw 520a and first screw 522a may be asymmetric, with one lead screw translating the carriage a greater distance than the other lead screw. The first and second screws may be in a generally stationary position relative to the carriage housing 512a. In some embodiments, there may be a transverse screw and transverse gear 530a which may provide a motive force to move the carriage in a transverse direction.

In various embodiments, the carriage housing may be an open frame structure, as shown, or it may be more enclosed. The blade assembly mount may be sterilized, and a partially open or mostly open structure may facilitate removal of biological waste, and allow for easier cleaning of the parts and assembly. In some embodiments, the design may be suitable for autoclaving. In some embodiments, the carriage housing may be part of a unibody design, where the front and back plates, and the support members may be a single unit, which may be machined, printed, assembled, stamped or manufactured by other means.

In an embodiment, a drive gear 510a may be coaxially engaged to either the second screw 520a or the first screw 522a. The drive gear 510a may be in mechanical communication with a motor or a translation gear, both of which are described herein. The carriage 514a may be driven by a single lead screw. In some embodiments, the individual lead screws may have a rotational gear on each lead screw, with a pinion gear in between the rotational gears. In this manner, one of the screws may serves as the drive axle with a driving rotational gear 508a, and torque may be distributed evenly to the other, non-drive axle screw through the pinion gear. The carriage housing 512a may be attached to, or mechanically engaged to the frame. The cap 506a may connect to a carriage 514a, and the carriage 514a may translate up and down on the axis defined by the first screw and also move the blade with the carriage. While some embodiments may use lead screws, other embodiments may use ball screws or any other screw-driven actuator. Equivalent mechanical actuators for moving the carriage may include linear actuators, rail and piston slides, cam shaft and so on. In various embodiments, the cap 506a, carriage 514a and blade 504a may be made as a single part, or multiple parts assembled together. In various embodiments, the dilator device may be made of various forms of stainless steel. In some embodiments the blades may be made from carbon fiber. In some embodiments the blades may be made of various alloys, including but not limited to titanium alloys, tungsten alloys or other steel alloys. In some embodiments the dilator device may be made of ceramic composite materials. In some embodiments various components of the dilator device may be made of polymers, such as poly carbonate, polyether ether ketone (PEEK) or polymer composites. It may be understood by those skilled in the art that various components of the dilator device may be made of different materials. In various embodiments, the dilator device may be reusable, so the materials selected for manufacturing the dilator device may be sterilizable, washable or cleaned.

In some embodiments, there may be a transverse screw that may be oriented in an orthogonal position relative to the first screw. The transverse screw may cause the carriage to move in a transverse axis, which may cause the blade portion to move in a transverse direction. In some embodiments, there may be more than one transverse screws, which may cause the blade to move in a variety of different directions.

In another embodiment, as shown in FIG. 5B, the blade assembly 500b may have a carriage housing 512b, with at a first screw 522b mechanically engaged to the carriage housing 512b. A carriage 514b may be in mechanical engagement with the first screw 522b, such that rotation of the first screw may axially displace the carriage 514b along at least a portion of the length of the screw 522b. The first screw 522b may have an additional gear 508b that may be used to drive a second screw. A blade 504b may be attached to the carriage 514b via a bridge 506b. The blade 504b may be positioned outside the housing 512b, while the carriage 514b may be positioned inside the housing 512b.

In an embodiment, the housing 512b may have an open architecture, which may promote cleaning the blade assembly after use. The open architecture may permit the parts within the housing to be washed or cleaned. In some embodiments, the blade assembly may have a second screw (not shown), which may also be mechanically engaged to the carriage housing 512b and the carriage 514b. In some embodiments, the second screw may intersect the carriage 514b at a substantially orthogonal angle to the first screw, so the carriage may be moved in a second direction different from the first. In various embodiments, the blade, housing, bridge and other components of the blade assembly may be made of stainless steel, aluminum, titanium or any metal alloy, polymer or ceramic composite. In some embodiments, a transverse screw and gear 530b may be provided to permit the carriage 514b to move in a transverse direction.

In some embodiments, the blade assembly may have one or more sensors. The sensor maybe any described herein, such as a force sensor, a strain sensor, a neurosensory, a magnetic sensor, electromagnetic sensor, and so on. In some embodiments, the blade assembly may have one or more markers. The markers may include a fiducial for optical detection, electromagnetic detection, or conductance. The marker may include a bar code, or a QR (Quick Response) code. The various sensor(s) and marker(s) may be placed anywhere on the blade assembly. In some embodiments, the sensor or marker may be positioned at the top or proximal end of the blade (near or on the bridge). In some embodiments, the sensor or marker may be placed along the length of the blade, or the distal end.

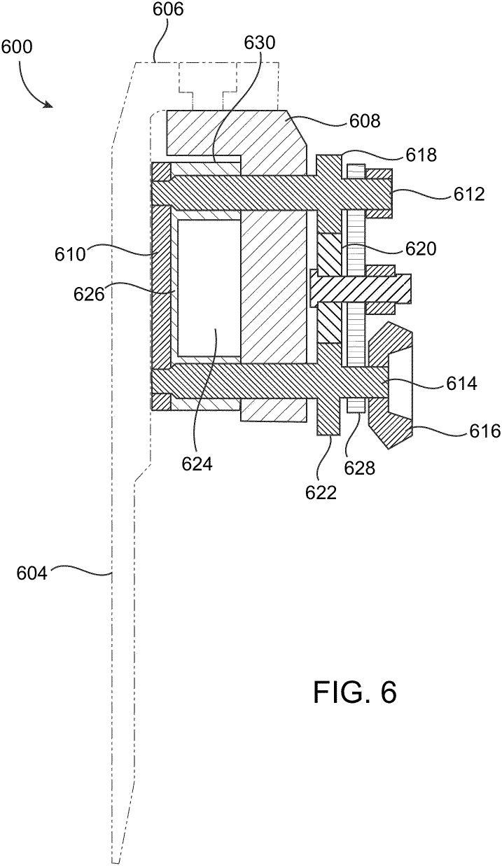

A cross section of the blade assembly mount 600 is now shown in FIG. 6. The blade (elongate member) 604 and cap 606 may be a unibody design, or individual components combined to form the design. The carriage 608 may be mechanically engaged to the cap 606 by a screw, weld or other fastener. In some embodiments, the blade 604, cap 606 and carriage 608 may be a unibody design. The carriage 608 may translate in and out on the upper lead screw 612 and lower lead screw 614. The lower lead screw 614 may be driven through the blade gear 616. Torque may be transmitted from the lower gear 622 to a pinion gear 620, and then to the upper gear 618. The carriage 608 may have a lip or flange on the top, for reaching over the top of the blade mount 630 and connecting to the cap 606. The carriage lip may be sufficient to bridge the distance of at least a portion of the gap space 624, the front end 626 of the blade mount and the width of the frame 610. In an embodiment, the carriage 608 may be fully translated backward so the blade may be in a fully open position, the carriage travels the furthest distance possible away from the frame 610. When the blade is in the closed position, the carriage may be in the closest position to the frame 610. While the present embodiment shows the blade gear 616 attached to the lower lead screw 614, it should be understood a single blade gear may drive the blade assembly from any one of the lead screws or pinion gear. Similarly, in some embodiments the carriage 608 may be driven using two or more blade gears on any combination of the upper and lower lead screws, and/or the pinion gear.

In various embodiments, the upper lead screw 612, the lower lead screw 614 may be supported by the back end 628 of the blade mount 630. In some embodiments, the blade mount 630 may be fastened to the frame 610. In some embodiments, the upper lead screw 612 and/or lower lead screw 614 may be in rotational engagement with the frame 610. In other embodiments, the upper and lower lead screws may be anchored to the frame. In still other embodiments, the upper and lower lead screws may terminate in the blade mount 630 without penetrating or touching the frame 610.

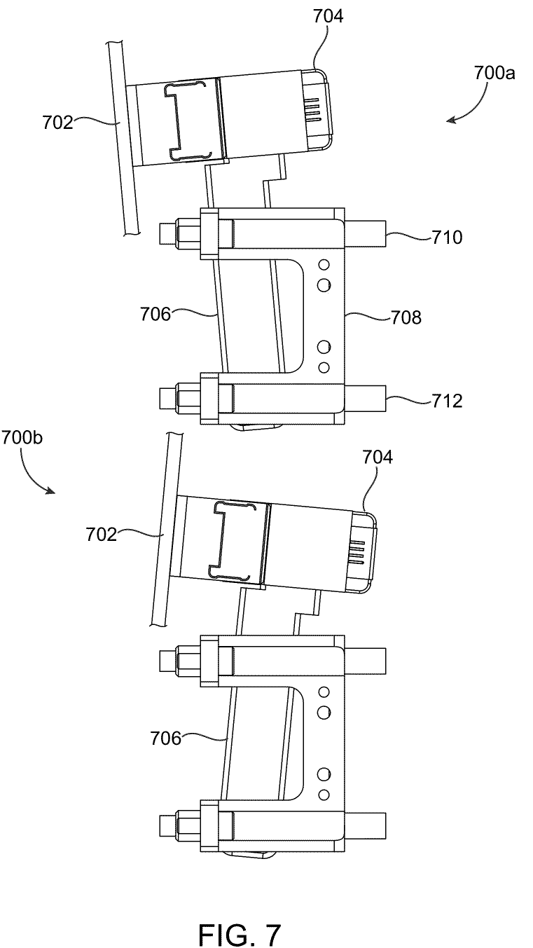

In some embodiments, the carriage of the blade assembly may be moved equally with both the upper and lower lead screws. In some embodiments, the displacement of the carriage on the upper and lower lead screw may not be equal, as shown in FIG. 7. In an embodiment, the upper lead screw 710 may move the carriage 706 a greater displacement than the lower lead screw 712, producing a tilt of the blade 702 so the proximal end of the blade may be tilted toward the center of the frame. In another embodiment, the lower lead screw 712 may displace the carriage a greater distance than the upper lead screw, causing the proximal end of the blade tilts away from the center of the frame. For reference purposes only, the images of FIG. 7 illustrate only a portion of the blade assembly. The frame is omitted.

In some embodiments, the carriage may be tilted in a side to side motion, causing the blade to move in an additional degree of freedom. The carriage may have a horizontal lead screw (not shown) that allows the carriage to be moved from side to side, in addition to back and forth. In some embodiments, the use of a horizontal lead screw in substantially an orthogonal relationship to the upper lead screw and the lower lead screw, may allow the carriage to move on an axis substantially orthogonal to the plane of the drawing page. In some embodiments, use of the different lead screws to control position of the carriage (and blade) may produce two axis of movement with four degrees of freedom (DOF). Use of a third axis parallel to the blade may produce a third axis and six DOF. In many embodiments, the dilator may be attached to a robotic arm, offering additional DOF with the use of the robotic arm.

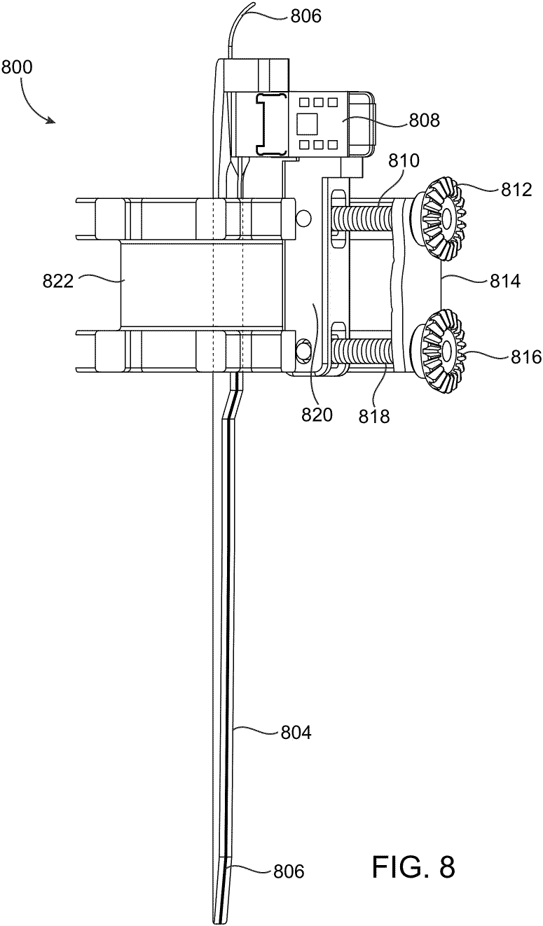

An angle view of a blade assembly is now shown in FIG. 8. In some embodiments, a blade assembly 800 may contain a blade 804 and a variety of support and mechanical structures to facilitate the motion of the blade. In some embodiments, a neuro sensor 806 may traverse the length of the blade (the blade is the elongate member of the tissue dilating apparatus). Note while the elongate member is referred to herein as a blade, the blade does not have a sharp edge. The blade is not designed to cut tissue or otherwise damage tissue by virtue of the shape of the blade. The blade instead is an elongate tool that is a component of a larger tool. As an analogy, each blade may be thought of as part of a straw cut into a number of equal sized pieces, and the straw being cut lengthwise. The straw may be cut into two or more lengthwise pieces. The pieces may be equal size in some embodiments. In other embodiments, the blade pieces may be unequal (asymmetrically cut) so some pieces may be larger or smaller than others. The lengthwise “straw” pieces may have atraumatic edges, with the convex portion of the straw body facing the tissue to be dilated.

In some embodiments, the blade 804 may be connected to a carriage 820. A force sensor 808 may be mounted on the blade to measure the force or strain exerted on the blade during use. An upper lead screw 810 may connect the carriage to an upper blade gear 812, and a lower lead screw 818 may connect the carriage 820 to a lower blade gear 816. A portion of the back end 814 of the blade mount (the rest omitted for clarity) may support the upper lead screw 810 and the lower lead screw 818. The blade mount back end 814 may be mechanically engaged to the frame 822 via a set of attachment points (not shown).

In operation, each of the upper blade gear 812 and the lower blade gear 816 may be rotated using a transition gear connected to a motor. The upper and lower gear may be rotated in synch or rotated individually. As the gears are rotated, the lead screws rotate and drive the carriage along the length of the lead screws forward or backward. If the two lead screws are rotated in unison, the carriage may maintain the same orientation from start to finish. In some embodiments the carriage may be orthogonal to the lead screws. In some embodiments the carriage may be tilted with respect to the lead screws. In other embodiments, the rotation of the upper and lower lead screws may cause the carriage to change its orientation. A higher linear displacement in the upper screw may cause the carriage to tilt forward (toward the frame), while a lower linear displacement of the upper lead screw compared to the lower lead screw may result in the carriage being tilted backward (away from the frame). In some embodiments, the blade 804 may be mechanically engaged to the carriage in a fixed orientation, with the carriage and blade in a substantially parallel alignment. In some embodiments, the frame 822 may be solid. In some embodiments, the frame 822 may have one or more openings along its perimeter.

In various embodiments, the blade 804 may have a tapered breadth. In some embodiments, the length of the elongate members/blades may be selected for the type of surgical procedure the dilator device may be used for. In some embodiments the length of the blade may be 40 millimeters (mm). In some embodiments the length may be 80 mm. In still other embodiments the length may exceed 120 mm. As the blade length becomes greater, there may be additional material used in the blade shape to provide structural integrity. In some embodiments, the proximal end of the blade (the end away from the surgical site) may be larger while the distal end (the end at or in closer proximity to the surgical site) may be narrower. In various embodiments, the blade may be tapered, or have a step down form with more material used in the proximal end. In some embodiments, the proximal end may be reinforced with stronger materials. In some embodiments, the blade may have structural support along its length, or portions of its length to provide enhanced structural rigidity and reduce the amount of flex the blade experiences under load.

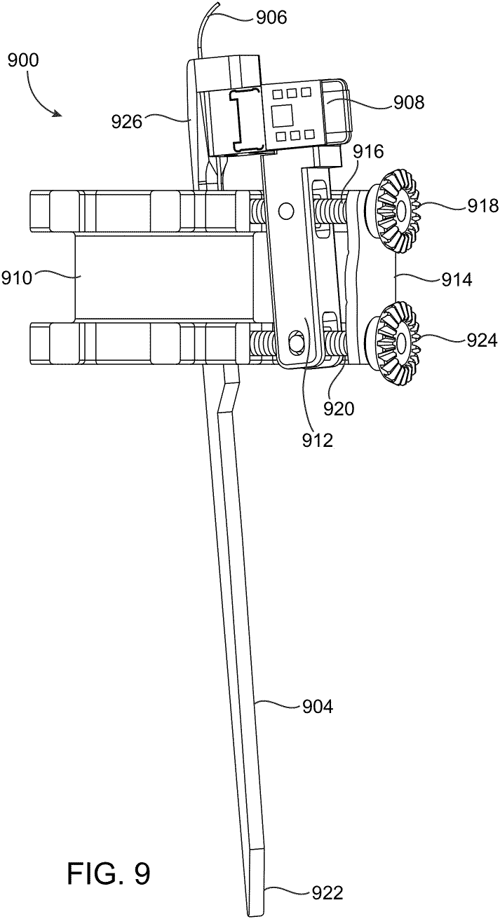

An example of a tilted blade is now shown in FIG. 9. In an embodiment, the blade assembly 900 may have a blade 904 with a neuro sensor 906. The blade may be mechanically engaged to the carriage 912, with a force sensor 908 attached to the blade. The upper lead screw 916 and lower lead screw 920 may drive the carriage 912 along the length of the upper and lower lead screws. The upper and lower lead screws may be supported by a back end 914. The back end 914 may be part of the blade mount (not shown for clarity), and the blade mount may be mechanically engaged to the frame 910. An upper blade gear 918 and lower blade gear 924 may operate to drive the carriage. In an embodiment, the blade proximal end 926 may be tilted toward the frame 910, causing the blade 904 to be tilted so the distal end 922 may be positioned further away from the center of the aperture (defined by the inner surface of the frame).

In an embodiment, the set of elongate members may come together to form a tapered dilator 1000 as shown in FIG. 10. The tapered dilator 1000 may have a wider dimension at the proximal end, and a narrower dimension at the distal end. The tapered dilator 1000 may have a set of blades 1004, that form an opening 1008 when the blades are in a closed position. The blades may have a strut 1006 down each blade to enhance structural rigidity. In some embodiments, the strut may be a neuro sensor for use in detecting nerve impulses rather than for structural integrity. In some embodiments the strut may be both a neuro sensor and a structural element. The closed position of the blades may form an opening, or an aperture, through which a surgical instrument may be passed down from the top of the dilator. Alternatively, a surgical instrument may be passed from the distal tip up to the proximal end, as when receiving an introducer or a guide wire.

In various embodiments, the closed position of the blades 1004 may be opened by actuation of a set of motors for driving the displacement of the carriage of each blade.

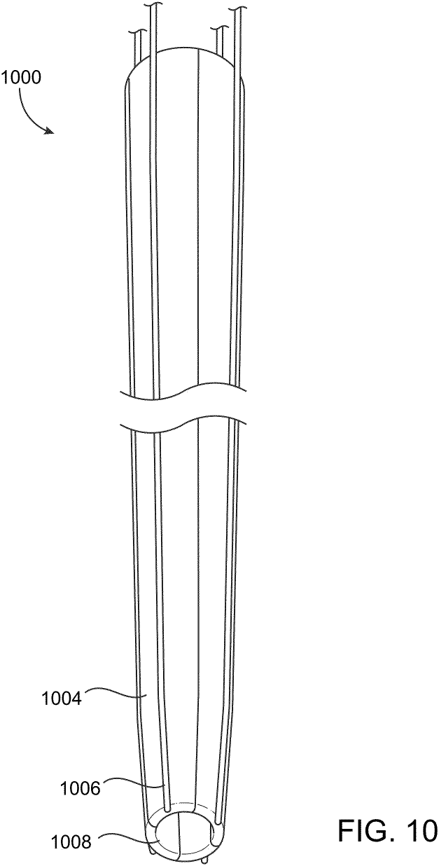

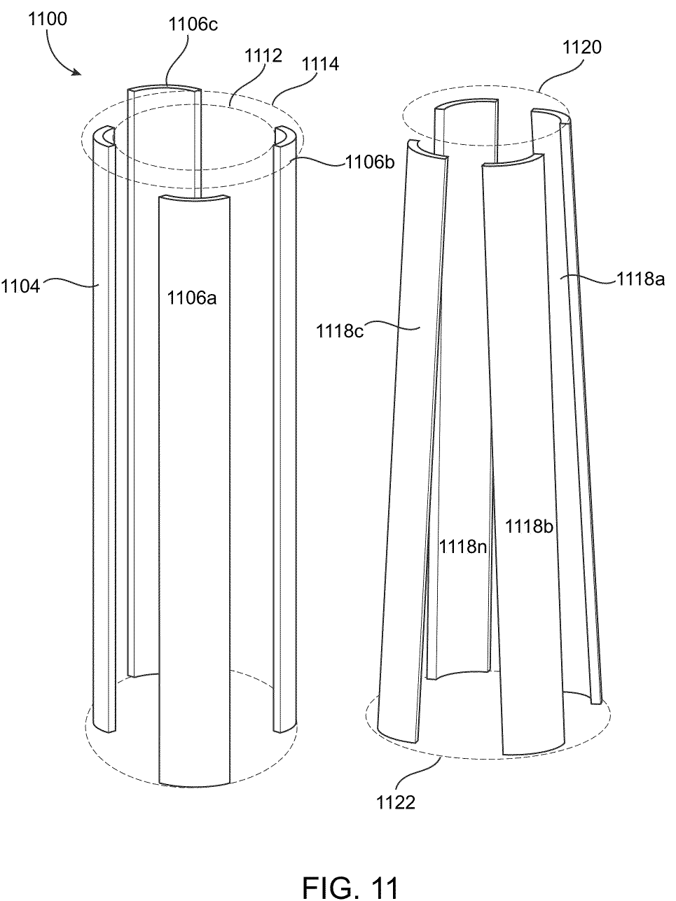

Examples of a parallel and non-parallel motion of the blades are now shown in FIG. 11. In an embodiment, four blades are illustrated as an example for defining a working channel. In an embodiment, the four blades are shown being parallel to each other. The working channel may have a closed footprint 1112, and an open footprint 1114. The open foot print may be an irregular shape, which may be the result of the blade members being opened in a non-uniform manner. In an embodiment, one blade member may remain in a closed position 1104, while other blade members may be in various stages of different open positions 1106_{a-c}.

In an alternative embodiment, the closed working channel may have a first foot print 1120, and then a second open foot print 1122. The individual blade members 1118ₐ₋ₙ may open in a tilted manner with the distal ends angled outward as shown. There is no limit to the variety and positions of the individual blade members and shapes of working channel footprints as is described herein.

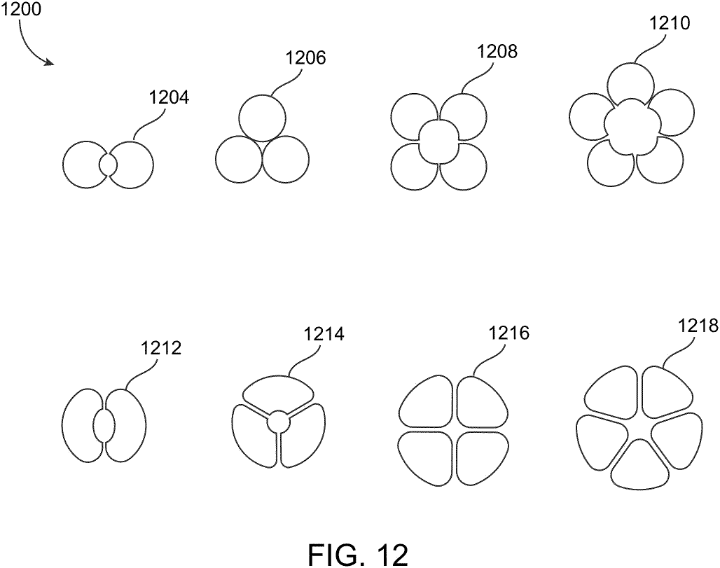

A variety of blade cross sections 1202 are now shown in FIG. 12. The set of elongate members may be any shape. In various embodiments, the cross section of the blades may be similar to the cross section of a tube. A tube may provide a working channel from the top of the tissue dilating device, to a surgical site. The tube may be used to introduce a guide wire, needle, or other long narrow device. The tissue dilator device may also be inserted over the long narrow device (such as an introducer), and the blades may be tapered near the distal end so the tissue dilating device may more easily be inserted into tissue over the introducer.

In an embodiment, the cross section of the blades may be circular (like round chopsticks), and placed as two members 1204, three members 1206, four members 1208, five members 1210 or more. In another embodiment, the elongate members may be formed to generally take the shape of sections of a circle (in cross section) like hemisphere 1212, triangle 1214, quarter 1216 or fifth 1218 pieces or more.

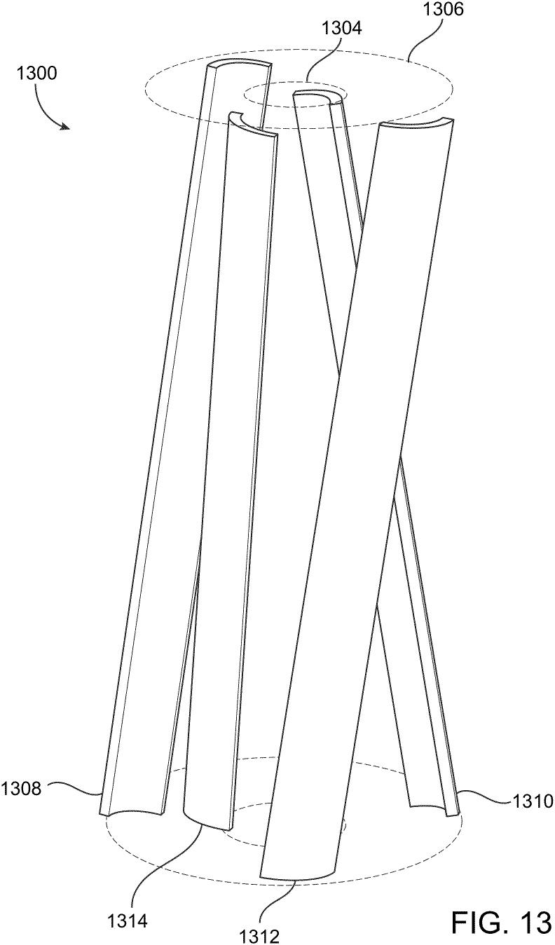

In an embodiment, there may be four blades that may be moved via their respective carriages to different positions 1300 as shown in FIG. 13. In an embodiment, there may be a closed perimeter 1304 and an open perimeter 1306 showing the limit of uniform movement of the four blade elements. When all four are closed, the four blades form a tube having a perimeter matching the closed perimeter 1304. When all four are open, they four blades expand outward until the blades lay on the open perimeter 1306. When the blades are not in the closed perimeter 1304 position, there may not be any connective material extending between the blades. In this fashion, when the blades expand, there is no contiguous material between the blade elements to create a circular working channel that is walled off between the outside and inside.

In some embodiments, the individual blades may be moved using a motor controller. Each blade assembly may have a force sensor, to measure the amount of force each blade is experiencing when moving against tissue. The motor controller may have force limits assigned to each blade, and these force limits may be uniform, or unique for each blade. When the motors drive each blade, the blades may not move in a uniform manner. The controller may deviate the motive force of each blade to “wiggle” the blades to create an opening that may also be a path of less resistance. The result is the first blade 1308, second blade 1310, third blade 1312 and fourth blade 1314 may produce an asymmetric pattern and pathway for a working channel. So long as the working channel is sufficiently sized to allow another surgical tool down the surgical channel, the tissue dilator may function as intended.

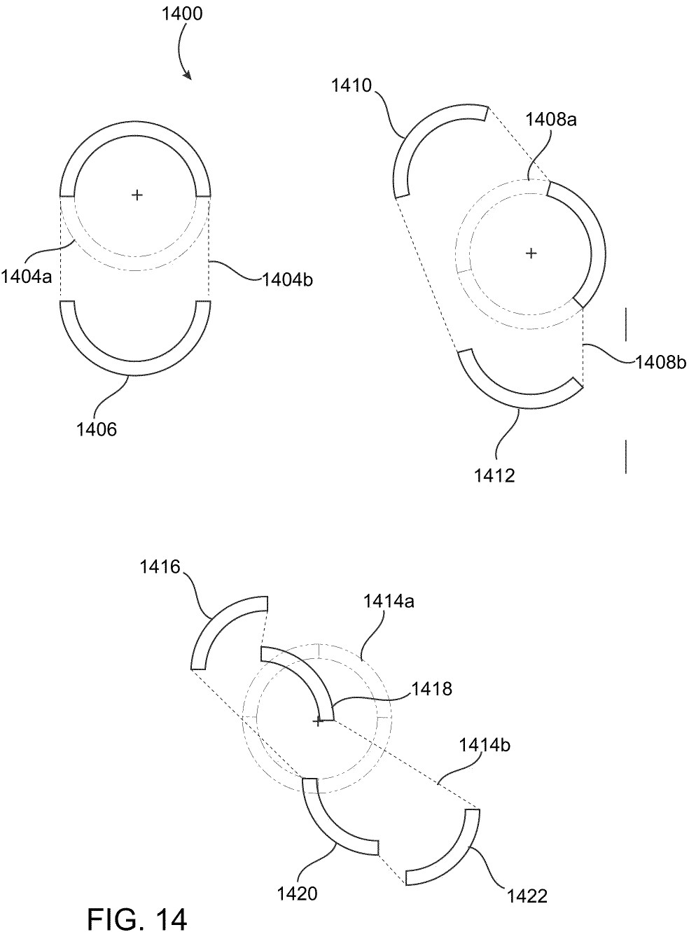

An end view of sample elongate member positions 1400 is now shown in FIG. 14. In an embodiment, there may be a two blade embodiment in a closed position 1404a shown as an outline, with one blade 1406 opening. The open position of the blade 1406 may change the working channel 1404b. In another embodiment, there may be a set of elongate members made of three blades with a three blade closed position 1408a shown as an outline. In an embodiment, two of the blades are in open positions, as one open blade 1410 and a second open blade 1412. The change in the position of the blades 1410, 1412 may cause the working channel 1408b to become larger. In another embodiment, a four blade closed position 1414a is shown as an outline. Then the first open blade 1416, second open blade 1412, third open blade 1420 and fourth open blade 1422 may produce a working channel 1414b a skewed pattern. The patterns shown in FIG. 14 are merely illustrative.

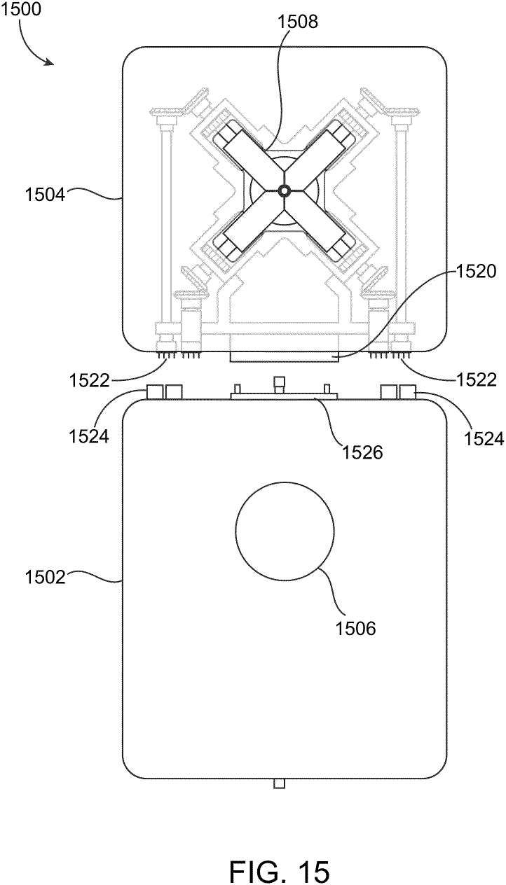

A dilator and motor 1500 are shown in FIG. 15. In an embodiment, the dilator casing 1504 can be seen from the top. The four blade caps 1508 can be seen external to the casing. When the individual blade assemblies open, the dilator creates a working channel for a surgical tool. A motor assembly 1502 may have motive gears 1524 to engage to the motor gears 1522 and an electrical controller 1526 to engage the electrical connector 1520. An access panel 1506 or port may be used to connect the motor assembly 1502 to a robotic arm (not shown).

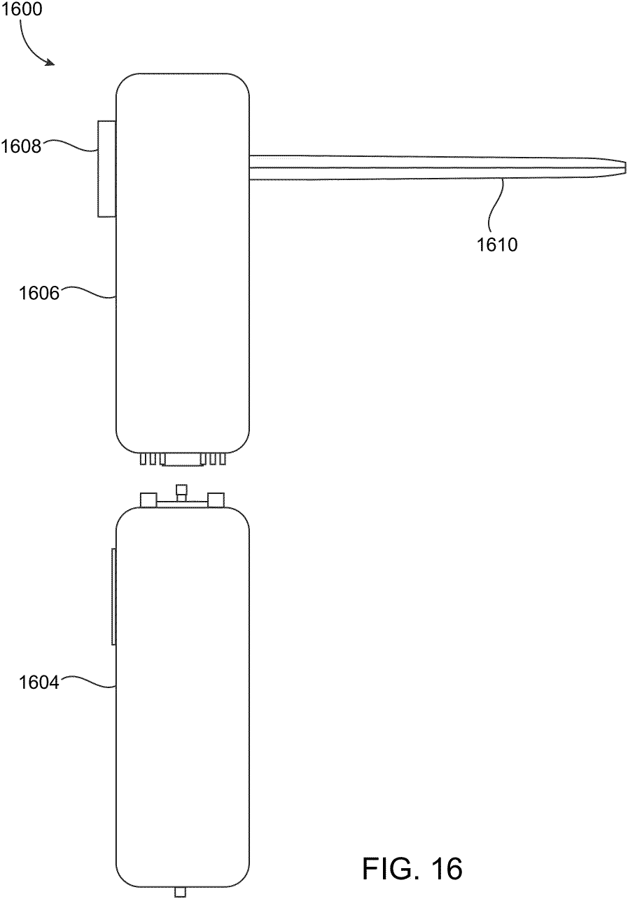

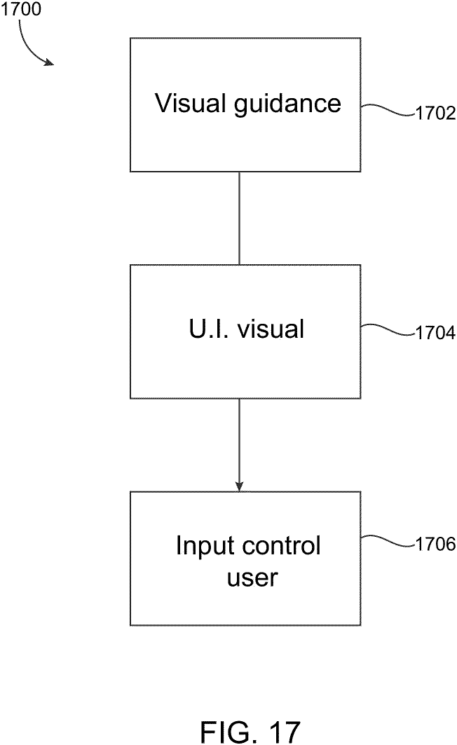

A profile view motor and dilator 1600 is now shown in FIG. 16. The set of elongate members 1610 are shown in a closed position, with the working channel collar 1608 shown on the opposite side of the dilator housing 1606. A motor housing 1604 may contain the motor assembly, which can mechanically engage the gear drive assembly protruding from the dilator housing 1606.

In some embodiments, a user may use a combination of visual guidance and robotic controls as a dilator controller 1700. In various embodiments, visual guidance 1702 may take a wide variety of forms. The visual guidance component may take images of the tissue to be operated on (including surrounding tissues used to access the desired surgical site, and tissues that may affect the surgery, as well as tissues the surgery may have an effect on). The scope of the visualization may be determined by preprogrammed parameters of the surgical system, the control system for the surgical dilator, the physician, by health care workers or even by insurance companies or policies.

In an embodiment, the visual guidance device may be a fluoroscopy device, with the fluoro images provided in electronic format to a computer. The visual guidance may also include a static or dynamic operation coordinate location system (a mapping system). The mapping system may use fixed positions external to the patient in combination with movable (dynamic) positions that are in or on the patient to create a three dimensional map of the surgical site, and track the position of the surgical tool, surgical site, and any objective tissues within the surgical site to be influenced by the surgical tool, or to be protected from influence of the surgical tool.

The fluoroscopic image (fluoro) may be combined with real time images using ultrasound, with the ultrasound images being integrated into the master imagery, which may be the fluoro image, or a computer interpolated (composite) image of the fluoro and ultrasound. In some other embodiments, nuclear magnetic resonance (NMR) images, positron emission topography (PET) scan images, or computer tomography (CT) scan images may be used with or instead of any other imaging modality described herein. Any future imaging modality which may provide guidance may also be used. The various imaging modalities may be used to produce a real time, or near real time display of the surgical site, and actual or representative images showing the penetration of surgical tools into the patient tissues. In some embodiments, the representation may not be a visual representation, but may be an audible signal, such as a change in frequency, amplitude, volume or elimination of sounds. A tactile feedback may also be used to alert or warn when the surgical tools are either approaching tissue that should not be interacted with, or target tissue that should be removed/altered.

In various embodiments, the visual images may be coordinated to produce an image map of the surgical area. Placement of the surgical tools into the image may be done using a registration system. The resulting image may form a user image visual 1704. The user may then interact with the surgical site by guiding the surgical tools, registered to the image of the surgical site, using one or more input controls for the user 1706. In various embodiments, the visual guidance system may be incorporated into a surgical robot and robot controller.

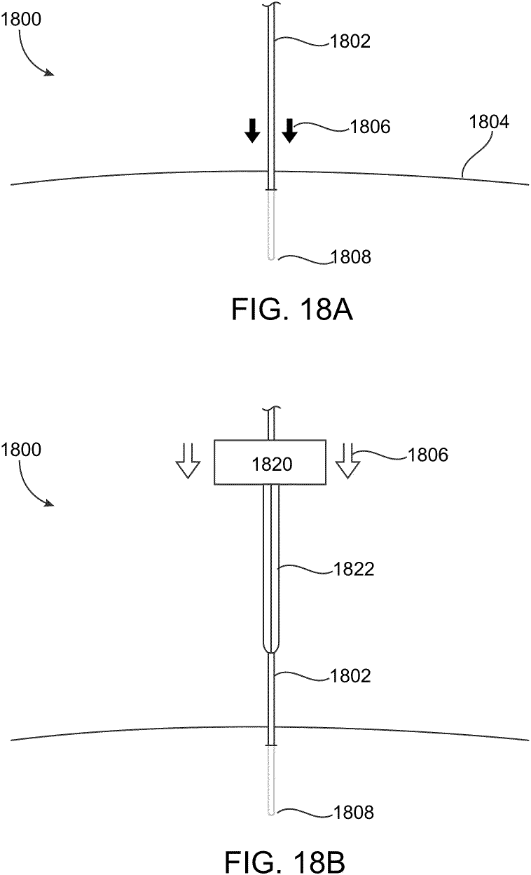

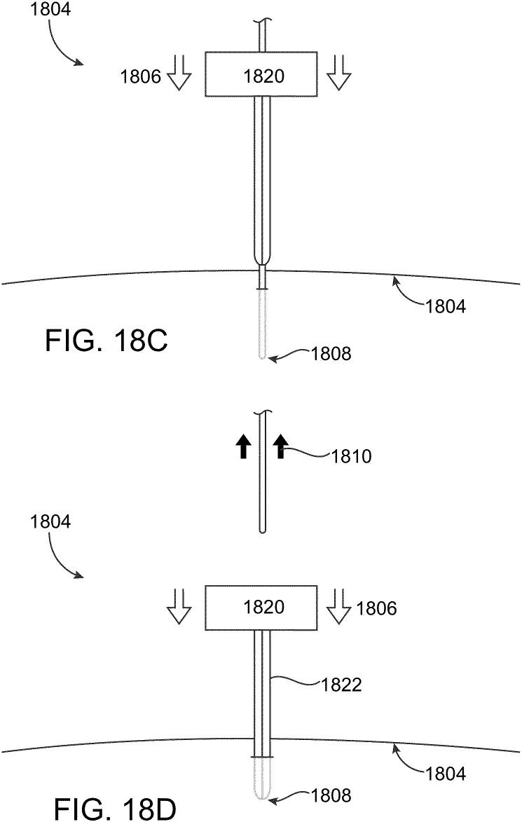

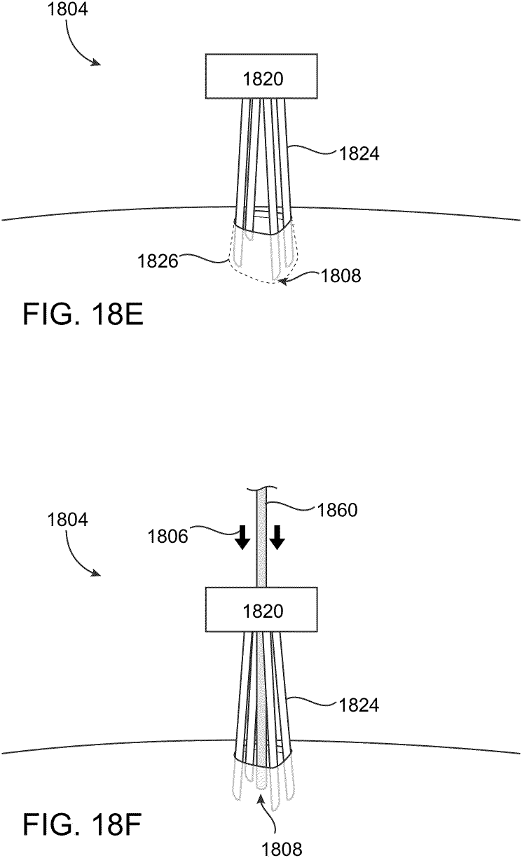

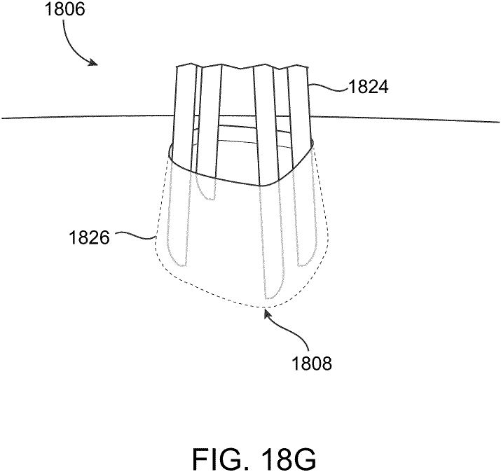

Several examples for the use of the dilator are now shown in FIGS. 18A-18G. In an aspect, of the method of use 1800, an introducer 1802 or other needle like insertion device may be pushed into a patient body. The point of entry may be a tissue line 1804 which may be the skin line, or may be a subdermal tissue such as muscle, interstitial connective tissues, fat and so on. In some surgical embodiments, an initial penetration may be made by non-robotic surgical techniques for a more general or gross exposure of internal tissues, and a fine or precise robotic approach may follow. In other aspects, a robotic approach may be made from start to finish.

The introducer 1802 may be pressed to a desired depth, with the tip of the introducer positioned at a depth of an operation site or surgical side 1808. The force arrows 1806 designate the direction of force used to apply the introducer. The introducer may be inserted manually, using robotic guidance, using full robotic control, or any combination of automated robotics and human guidance, as well as robotic forces and human forces, as may be appropriate for the procedure.

In an embodiment, once the introducer may be positioned, a dilator 1820 as described herein, may be slidably advanced over the introducer 1802 as shown in FIG. 18B. The closed members of the set of elongate members may form an aperture of suitable diameter to slide over the length of the introducer without binding, and without excess play in the mechanical fit of the two devices. The set of elongate blades 1822 may be advanced over the introducer 1802 until the tip of the elongate blades 1822 may be in sufficient proximity to the operation site 1808 as determined by the position of the introducer tip.

The dilator 1820 may be advanced over the introducer 1802 as shown in FIG. 18C. The dilator 1820 may be pushed into the tissue 1804 to the same depth as the introducer. In some aspects, the dilator 1820 may be pushed somewhat less or somewhat more, than the depth of the introducer.

FIG. 18D now shows the dilator 1820 may be positioned at the depth of the operation site 1808, the dilator or introducer 1802 may be withdrawn along the opposite direction 1810 of insertion. In some embodiments, the dilator 1820 may have some amount of continuing force 1806 applied to it to maintain its position with respect to the surgical site 1808 and/or the patient tissue line 1804.

FIG. 18E. now shows the dilator has been removed, and the set of elongate members may be opened to form an open member configuration 1824. The members may maintain their position at the surgical site 1808, and create a working channel 1826.

FIG. 18F. now shows the insertion of a surgical tool 1860 through the aperture of the dilator and between the open elongate members 1824.

FIG. 18G no shows the working channel 1826 created by the elongate members 1824 at the surgical site 1808. The working channel 1826 may be created to be large enough for a surgical tool to work in the surgical site. The surgical tool may have its own visualization capability (such as an endoscope), or rely on registration with the surgical site map, and visual presentation on a computer screen.

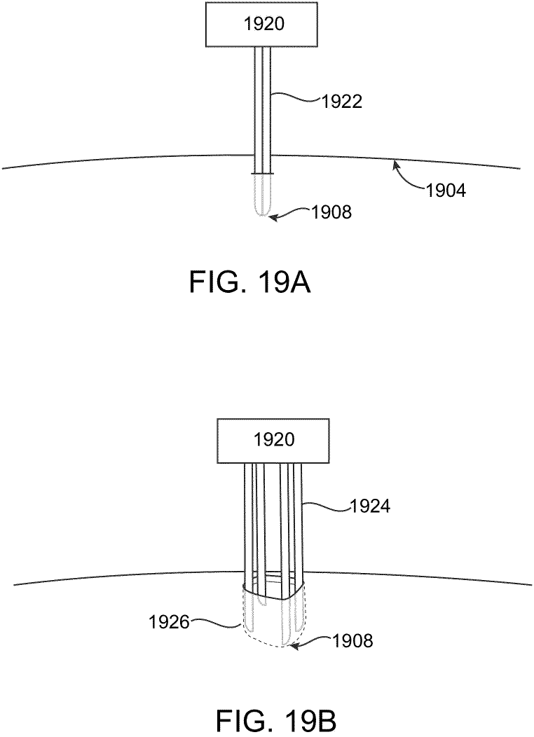

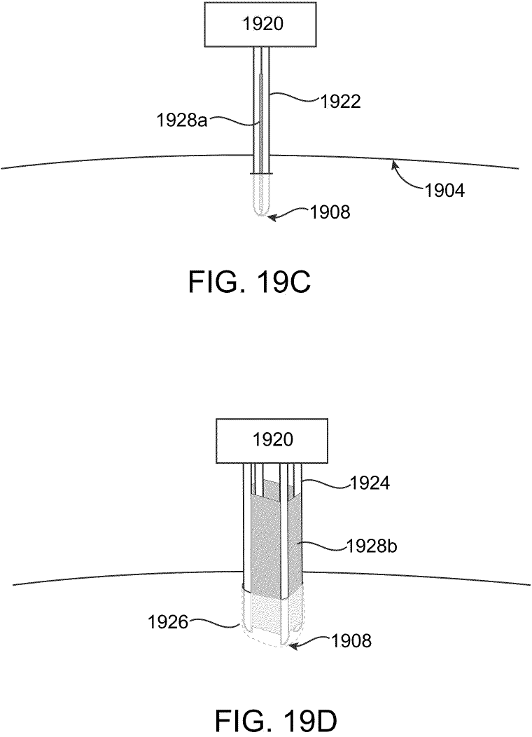

In another example use case, the elongate members may remain in a parallel configuration when used as shown in FIGS. 19A-19B. In an embodiment, the tissue dilator 1920 may be inserted to a surgical site 1908 as previously described. The blades 1922 may be in a closed configuration during insertion (FIG. 19A). The tissue line 1904 may be any tissue to be dilated, including skin, muscle, fat, subcutaneous tissue and so on. When the blades are actuated, the blades may spread apart in a programmed fashion, or manually manipulated, or a combination of the two. In some embodiments the blades may use a position control method. In some embodiments the blades may move using a force (torque) control method. Once the blades may be sufficiently opened to create a working channel 1926, a surgical tool (not shown) may be inserted through the dilator 1920 to access the surgical site (FIG. 19B).

In another embodiment, the elongate members may be connected together on the distal ends using a flexible or expandable structure as shown in FIGS. 19C-19D. In an embodiment, an expandable material 1928a may be in mechanical engagement with the elongate members or blades 1922 as shown in FIG. 19C. When the elongate members expand to form the working channel 1926, the expandable material 1928b may form the perimeter of the working channel. In some embodiments, the expandable material may connect the blades along the full length of the blades. In some embodiments the expandable material may connect the distal ends of the blades, or that portion of the blades that may be in contact with the patient tissue.

In various embodiments, the expandable material may be a stretchable material such as an elastic polymer, natural rubber, latex or similar material. In certain embodiments, the expandable material may be a mesh or netting material that may be porous, but of sufficiently fine mesh to prevent or reduce the intrusion of solid tissue into the working channel. In other embodiments, the exterior surface of the expandable material may be treated with a hydrophobic coating, to assist in repelling fluids from the working channel. A wide variety of materials may be used for the expandable material, including, but not limited to, polymers, ceramics, fabrics, metals, alloys or any combination of useful materials. The expandable material may be reusable or disposable.

In some embodiments, the material may form a helically wound braid (HWB) which may tighten as the braid expands. The initial form of the helically wound braid may be relatively loose fitting. The helically wound braid may be attached, or may be engaged by, the elongate members. In an embodiment, the helically wound braid may be woven around the elongate members, so the HWB may be long and thin during deployment. Then as the elongate members expand, the HWB may shorten and the braid may compress, forming a barrier against tissue or fluid intrusion into the working channel. In some embodiments, the HWB may have engagement loops which may be engaged by the elongate members. The HWB may be inserted into a surgical site over an introducer, and the tissue dilator may be inserted over the introducer following the HWB. The elongate members of the tissue dilator may engage with the HWB and deploy the HWB as the elongate members expand.

In some embodiments, a control system or control method may be used to control the tissue dilating apparatus. In some embodiments, the control system may take advantage of various sensing and actuation capabilities of the dilator system to realize different control strategies. On a high level, the control system may engage the actuation force and/or control the position of each blade. The system may monitor the blade position, the forces exerted on each blade, as well as measure positions of relevant structure in the surgical environment (such as neuro-monitoring signals to determine the location of nerves). The measuring of forces on each blade, and the measurement of the environment around each blade may be done continuously, or with a sufficient sampling rate to promote safety, with all data fed back to the control system.

In an embodiment, there may be a control method that uses position control. A user may specify the desired center axis and diameter of a planned working channel. The control system may convert these parameters from a user coordinate system into the dilator coordinate system, and then use a position based feedback control loop to servo each blade to the desired position to establish the desired working channel. Furthermore, the user may also adjust the individual position of each blade, for a more customized shape and size of the working channel.

In an embodiment, the position control method may be used together with force sensing and neuro monitoring. For example, if the tissue interaction force is below a specified threshold and/or neuro-monitoring signal indicates no critical neural structures are in the vicinity, the blade may be moved with a standard speed; when the force increases above a first threshold, or neuromonitoring shows the blade may be close to a nerve root, the blade speed may be reduced and warning signals (visual, auditory, haptic, etc.) may be used to alert the user. In some embodiments, if the force exceeds a second threshold that may be deemed unsafe, or neuromonitoring measures the blade may be too close to a critical structure, the blade motion may be stopped or even deflected, and the user may be informed with these warning signals.

In another embodiment, a force control system may be used. In an embodiment, each blade may be moved to reach a target force value or to follow a prescribed force profile (e.g., a force-time curve). Compared to the position control embodiment, the force control system may allow the maximum opening given a force threshold. This method may also be used together with position and neuromonitoring feedback. For example, if there are additional position-based constraints, such as a forbidden region predefined with a given preoperative image position, or with real-time surgical navigation, or with neuromonitoring, these constraints may be simulated as opposing forces to prevent the blade from violating them. Similar to the position control scheme, various warning signals may be used to alert the user.

In another embodiment, there may be a position and force hybrid control method. In some embodiments, when the dilator blades may move in an open or close translation direction, a position control may be used to reach a predefined opening. In another embodiment, such as tilting the blade, force control may be used to create a conical, cylindrical or asymmetric workspace while maintaining pressure force on tissue below a safe limit. Besides hybrid control for each blade, hybrid control may be employed across different blades, i.e., some blades may be controlled with a primary position feedback loop, while other blades may be controlled with a primary force feedback loop. In some embodiments, the shape and size of the working channel may be restricted by the patient anatomy, and the control system may adapt to limitations resulting from those restrictions.

An example of a control method is now shown in FIG. 20. In an embodiment, a control method 2000 may begin with a reference command 2002. The reference command may instruct the control system to move one or more blades, identified as a->n, to a particular position, move with a particular force, assume a particular shape, or move tissue along a particular path. A controller 2004 may control a motor, and using one or both of torque (force) control 2006a or positions control 2028a, may cause the actuation 2008a of a first blade 2050a. The system may monitor the torque 2020a and position 2012a of the first blade 2050a as a result of the actuation 2008a. The blade 2050a may then move in response to the actuation 2008a, but have its motion limited by opposing forces 2016a caused by resistance from the patient anatomy 2018. The blade 2050a may provide feedback to the controller 2004 in the resistance force 2024a it experiences through a force sensor as described herein. The blade 2050a may also have a neural sensor to detect neural signal 2026a in close proximity to the blade 2050a.

The process of controlling a second blade 2050b may follow the same control and feedback pathways, with a motor torque 2006b and position control 2028b provided to a motor to produce actuation 2008b. The actuation reports back the torque provided 2020b and the position 2022b. The second blade 2050b may also experience resistance force 2016b caused by the patient anatomy 2018. The blade may report data back to the controller 2004 in the form of resistance forces 2024b and any neural signal 2026b detected.

The process may be duplicated for each blade used to make up the dilator, as shown, up to N number of blades.

In another embodiment, there may be a method of controlling a tissue dilating apparatus having two or more independently operable blades. The method may comprise providing a reference command to a controller. The method may include determining, by the controller, a position and an amount of motor torque to relay to a motor, where the position may be determined from the reference command. The method may include actuating a motor, to create the amount of motor torque to move a blade to the position. The method may include registering, from a sensor, the resistance force on the blade, and then compensating using the controller, for the resistance force on the blade, by further actuating the motor. The compensating may be continuous and done in real time, as the blade may have a force sensor to determine the resistance of the tissue, as well as a location sensor, to determine the blade location in the surgical space.

The method may be used to control each blade individually, with an overall control program to monitor each blade’s movement relative to the reference command. The overall control program may determine alternative motions for each blade as a potential compensation to one or more blades that may encounter strong resistance to their initial movement instructions. The alternative motion may be executed by the overall control program automatically, or recommended to the surgeon for approval. In various embodiments, the overall control program may monitor the position of all blades and provide either direct changes or recommendations to change, the movement path of any blade, based on the medical procedure or other parameters available to, or programmed into, the overall control program.

In an embodiment, the reference command may be input by a surgeon during a medical procedure. The surgeon may be physically present with the patient, or may be remote (not in the same room as the patient or the dilator/surgical robot). The reference command may be continuously updated, as when a surgeon may be engaged in a surgery or medical procedure, and need to move the dilator continuously, or during certain periods of time. The reference command may be a pre-programmed maneuver of the control software for the dilator, or for a robotic surgical system used to control the dilator.

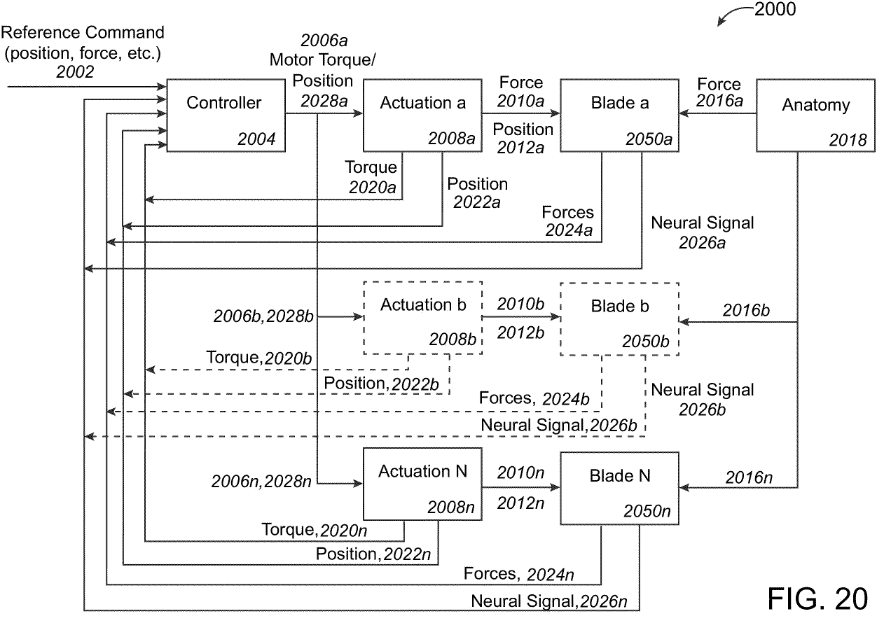

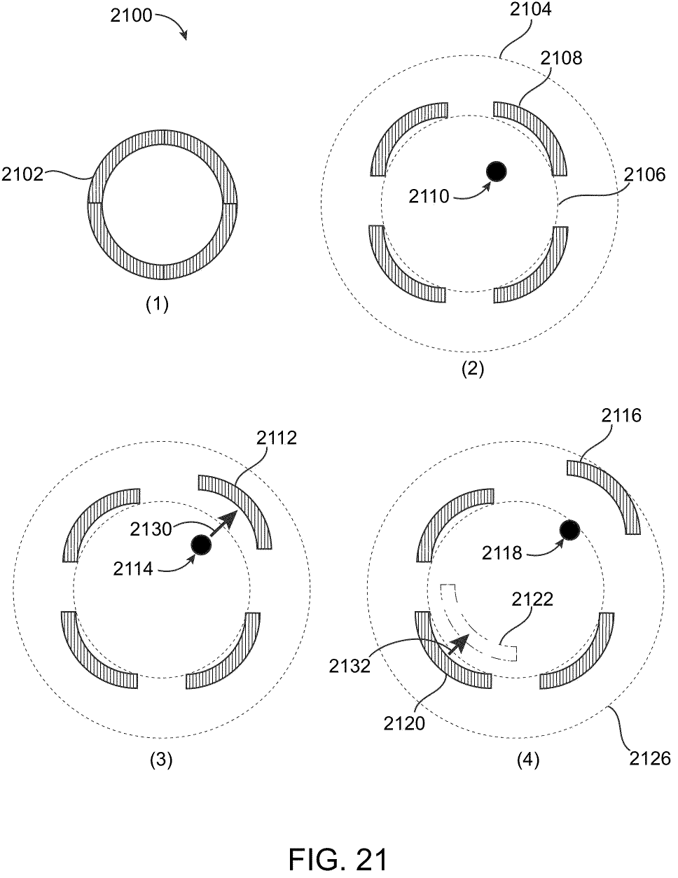

Example blade movement using the different control methods are now shown in FIG. 21. In various embodiments, the different control methods may be used at different stages during surgery. In an embodiment, the position based control method may be used to establish the working channel at the beginning of a surgical procedure. The workspace may be dynamically adjusted later, as the surgeon may use surgical instruments to operate in the working channel. In an embodiment, the dilator may have an initially closed position 2102. The view of FIG. 21 may be thought of as looking at a cross section of the distal end of the blades, or looking at the distal tip of the blades in a “head one” view. The shape of the blades presented is merely illustrative, and should not be taken as limiting for the actual shape or cross section of the blades.

In an embodiment, the dilator blades may open to a second diameter 2104 using the position control method. An instrument 2110 may then be positioned in the dilator. The initial dilator diameter is shown with a first diameter 2106. The second, expanded dilator diameter is shown with a second diameter 2104. The individual blade tips 2108 are positioned about the first diameter with the medical tool 2110 disposed within the first diameter 2106. In an embodiment, the force control method may be adopted. The surgical instrument may be moved toward the top right blade 2112, and the blade may be “pushed” by a virtual force 2130 rendered from the proximity between the surgical instrument 2114 and the blade 2112, causing the blade 2112 to “open up” (move further to the top right) to allow additional workspace for the instrument. As the blade moves toward the limit of the second diameter, the blade 2116 may reach a “hard stop” of the second diameter limit 2126. The surgical tool 2118 may move as close to the blade 2116 as may be physically possible, or delineated by a control limit of the dilator control method. In some embodiments, the lower left blade 2120 may shift 2132 toward the center, to a new position 2122, as shown. In this manner, the lower left blade 2120 may reduce force on the opposing side such that the likelihood of tissue injury may be reduced. This may be done in either force or position control methods.

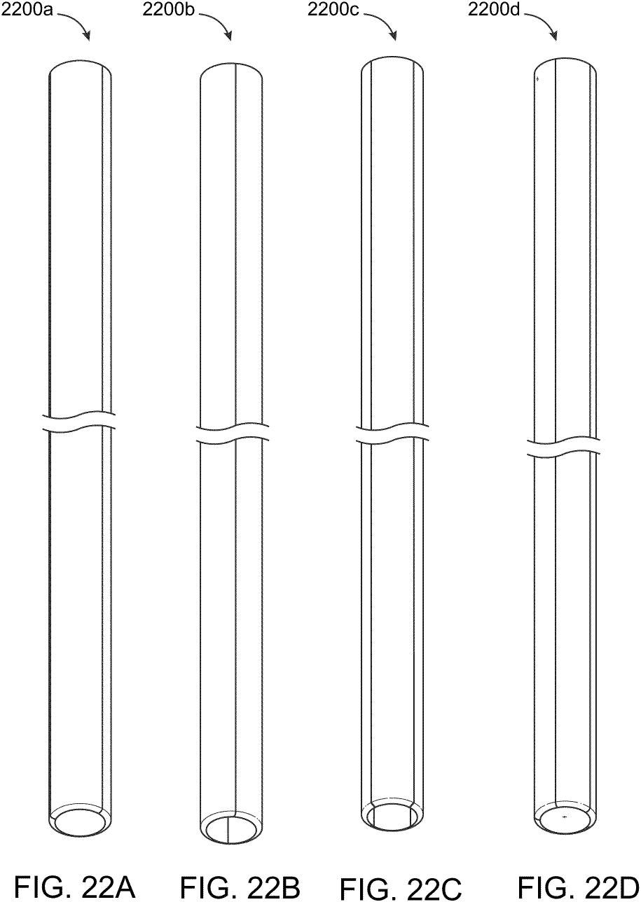

Additional embodiments of differing numbers of blades for the dilator are illustrated in FIGS. 22A-22D. In various embodiments, an example of a three blade dilator is illustrated in FIG. 22A, an example of two-blade dilator 2200b is illustrated in FIG. 22B, an example of four-blade 2200c is illustrated in FIG. 22C, and an example of five-blade dilator is illustrated in FIG. 22D.

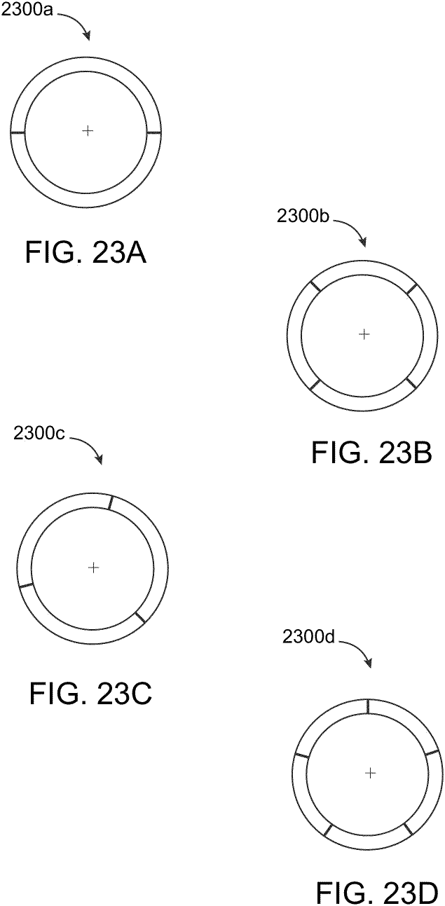

The cross section views of additional embodiments of differing numbers of blades for the dilator are illustrated in FIGS. 23A-23D. An example of a two-blade dilator is illustrated in FIG. 23A. An example of a four-blade dilator is illustrated in FIG. 23B. An example of a three-blade dilator is illustrated in FIG. 23C. An example of a five-blade dilator is illustrated in FIG. 23D.

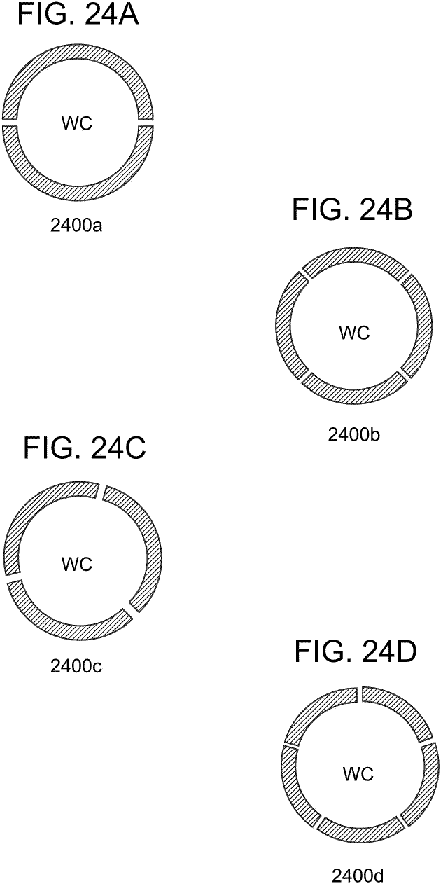

Various blades (e.g., blades illustrated in FIGS. 23A-23D) in closed positions and the potential regular layouts of example working channels 2400a, 2400b, 2400c, and 2400d are shown in FIGS. 24A-24D.

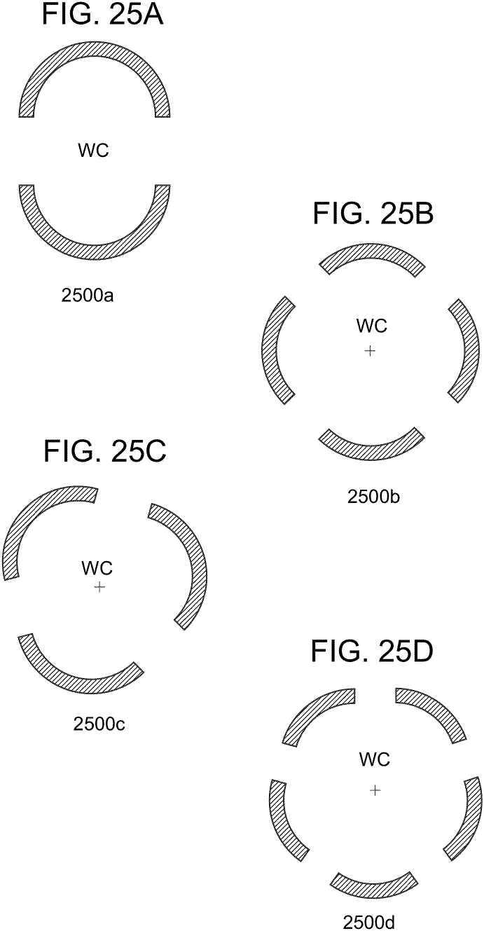

The opening of the various blades (e.g., blades illustrated in FIGS. 23A-23D) and the potential regular layouts of example working channels 2500a, 2500b, 2500c, and 2500d are shown in FIGS. 25A-25D.

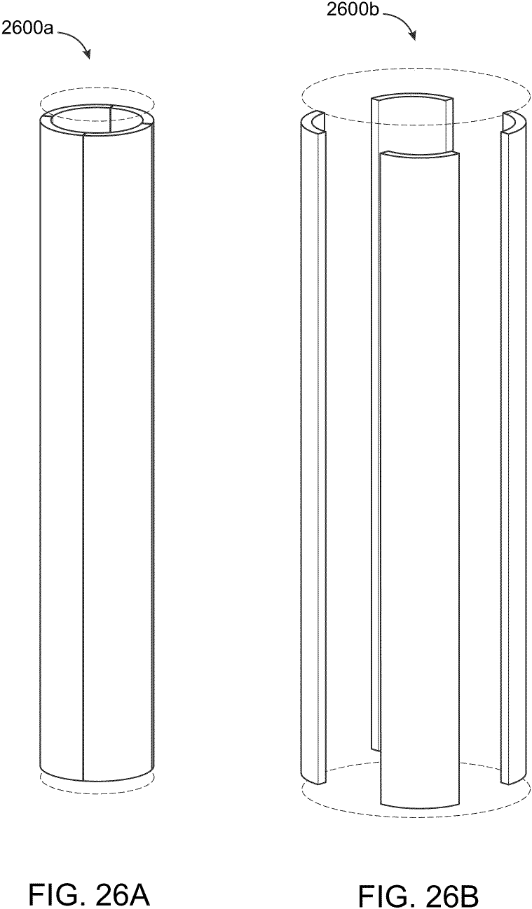

In an embodiment, a view of a closed set of blades 2600a is shown in FIG. 26A, and a view of an open set of blades 2600b is shown in FIG. 26B. In some examples, the sets of blades retain a substantially parallel configuration with respect to each other while moving.

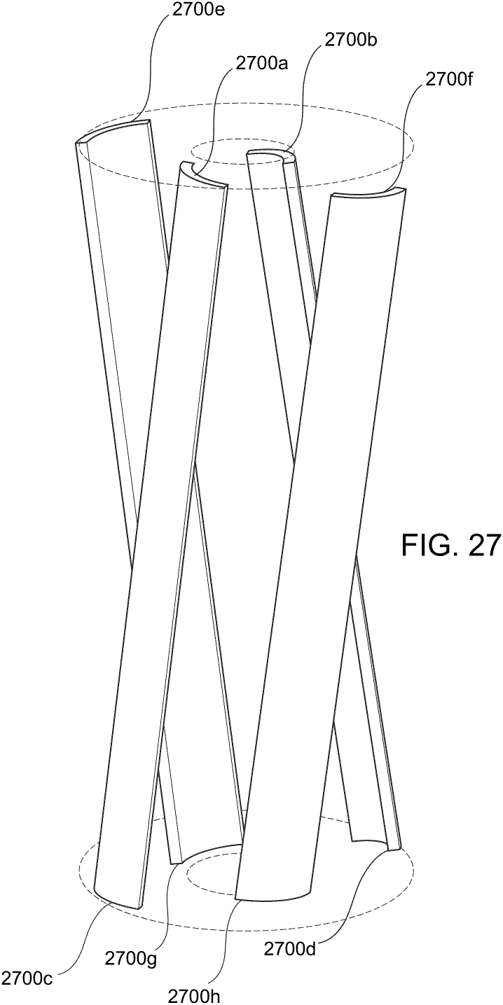

In an embodiment, a view of four blades in different positions are now shown in FIG. 27. The four blades may be individually movable in pairs. Here two blades are shown with their proximal ends 2700a and 2700b near the closed position as seen at the top, and open positions at their distal ends 2700c and 2700d. In opposition the other two blades are shown with their proximal ends 2700e and 2700f spaced far apart, while their distal ends 2700g and 2700h are close together near the closed position.

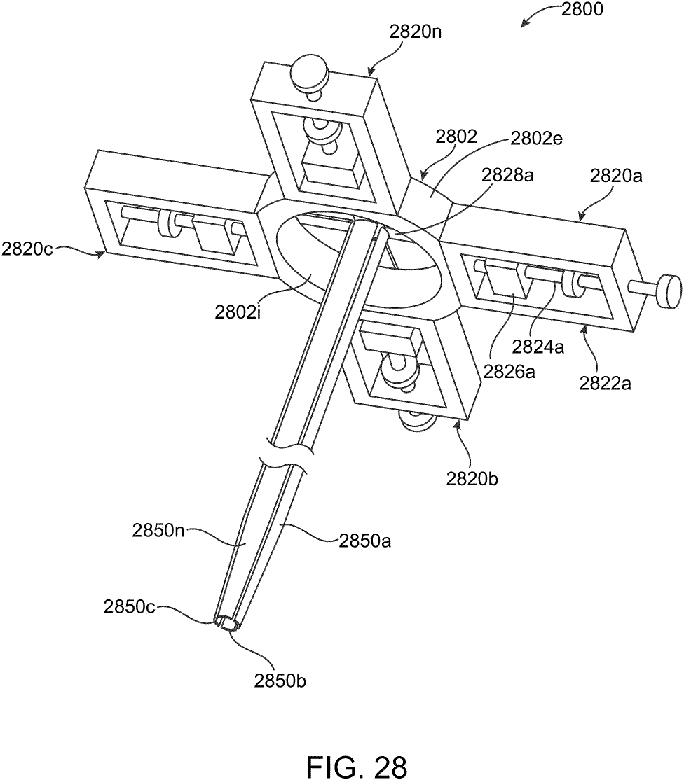

In some embodiments, there may be a tissue dilating apparatus for use with a robotic surgical system as shown in FIG. 28. In an embodiment, the tissue dilating apparatus 2800 may have a frame with an interior 2802i and an exterior 2802e. The apparatus may have a plurality of blade assembly apparatus 2820a-n as described herein. Each blade assembly may be mounted to the exterior of the frame. Each blade assembly may have a housing 2822a, a first screw 2824a mechanically engaged to the housing 2822a, a carriage 2826a, and a blade 2850an. The carriage 2826a may be mechanically engaged to the first screw 2824a, such that rotation of the first screw 2824a can displace the carriage 2826a along at least a portion of the length of the screw 2824a. As described herein, the first screw may be a lead screw, ball screw or standard screw, with the carriage having the appropriate receptacle for the type of screw used. The blade 2850a may be fixedly attached to the carriage 2826a using a bridge 2828a or spar, to bridge the distance between the mount and the frame 2802, so the carriage 2826a may be in the housing 2822a of the blade assembly while the blade may in the within the space defined by the frame interior 2802i. When the first screw 2824a rotates, it causes the displacement of the carriage 2826a, moving the carriage along the axis of the first screw. The first screw 2824a may be mechanically engaged with the housing and/or the frame. In an embodiment, the screw generally does not change its axial displacement relative to the frame or the housing. A number of actuators matching the number of blade assemblies may be used to drive the screws causing the carriages to move.

In an embodiment, the first screw may pass through the frame, and engage the blade directly. The screw may control the radial motion of the blade (toward and away from the center). This may be done by having the screw terminate in or on the blade, and the blade move as the screw is rotated. In some embodiments, the screw may be replaced by a piston, slider or other linear movement device. In some embodiments the screw may be an arm attached to a cam, converting rotational movement to linear movement.

In some embodiments, the tissue dilating apparatus may have two or more blades that may come together to form a tube. The blades may be individually moveable. The dilator may also have a group of motors in removable mechanical engagement to the mechanical actuators. Generally, there may be one motor to drive one mechanical actuator, and in turn drive one blade assembly. In some embodiments, the motor drive may be directly engaged with the blade assembly. The tissue dilating apparatus may be made of stainless steel, aluminum, or medical grade metal alloys.

The shape of the blades may depend on the number of blades used to create the working channel of the dilator. Referring to FIGS. 24A-D and 25A-D, in some embodiments, where two blades are used, each blade may have a semi-circular cross section and collectively form a closed working channel WC 2400a when closed, and an open working channel WC 2500a when open, as illustrated in FIG. 24A and FIG. 25A. The blades define a working channel WC. In another embodiment, three blades may be used. The cross section of the closed position blades may define an enclosed working channel WC 2400b, while the cross section of the open position blades may partially define a partially open working channel WC 2500b, the blade cross sections may be similar to FIG. 24B when the blades are closed, and FIG. 25B when the blades are open. In another embodiment, there four blades may be used, the cross section of the blades may be similar to that shown in FIG. 24C when the blades are closed to form a closed working channel WC 2400c, and FIG. 25C when the blades are open to form an open working channel WC 2500c. In still another embodiment, there may be five blades used for the dilator with a cross section of the five blades as shown in FIG. 24D when the blades are closed to form a closed working channel WC 2400d, and FIG. 25D when all five blades are open to form an open working channel WC 2500d. The area of space between the open cross section blades in FIGS. 24A-24D and 25A-25D illustrate the working channel created by the dilator blades.

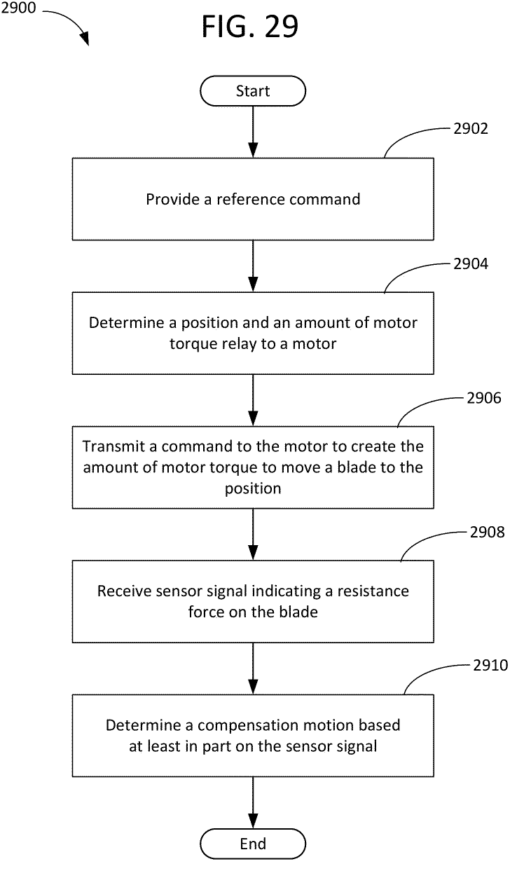

FIG. 29 shows a simplified flow diagram showing a method 2900 for using a dilator according to embodiments disclosed herein. This diagram is merely an example. One of ordinary skill in the art would recognize many variations, alternatives, and modifications. The method 2900 for using a tissue dilator includes processes 2902, 2904, 2906, 2908, and 2910. Although the above has been shown using a selected group of processes for the method 2900 for using a tissue dilator, there can be many alternatives, modifications, and variations. For example, some of the processes may be expanded and/or combined. Other processes may be inserted to those noted above. Depending upon the embodiment, the sequence of processes may be interchanged with others replaced. Further details of these processes are found throughout the present disclosure.

In some embodiments, the tissue dilating apparatus has a plurality of independently operable blades. In process 2902, a controller provides a reference command. In process 2904, the controller determines a position and an amount of motor torque relay to a motor, wherein the position is determined based at least in part upon the reference command. In process 2906, a command is transmitted to the motor to create the amount of motor torque to move a blade to the position. In process 2908, the controller receives the sensor signal (e.g., one or more sensor signals, a plurality of sensor signals), for example, from one or more sensors, where the sensor signal indicates or can be used to determine a resistance force on the blade. In process 2910, the controller determines a compensation motion (e.g., actuation, expansion, rotation, etc.) based at least in part on the sensor signal.

In some examples, the controller moves the plurality of independently operable blades to create a working channel for a surgical instrument. In some examples, the plurality of independently operable blades form a tube. In some examples, the sensor is a force sensor configured to measure the resistance force applied by tissues to the blades as the working channel is created. In some examples, the compensation motion may be determined continuously and in real-time (e.g., less than 1 second) by the controller.

In the many embodiments, the blades may define a portion of the working channel for a surgical procedure. The blades (which are atraumatic) may be used to push tissue out of the way, and clear a path for a working channel. If the tissue may be pushed aside using the blades, the working channel may be created without any additional device or effort. In some embodiments, the tissue may be spongy, wet or bleeding, which may spill into the working channel. In embodiments where the blades may not be sufficient to define a working channel by themselves, additional elements may be introduced into the surgery, such as a dam, a flexible wall, such as made of medical grade rubber or polymer, or a helical wound cuff or collar of medical grade material. This additional dam or barrier may be stretched over a portion of the blades (such as near the distal tips), or may be erected around the blades before, during or after creation of the working channel.

### Aspects

The disclosure of the tissue dilating apparatus is now presented in a series of non-limiting aspects:
1. A blade assembly comprising:
   a mount comprising a back end, a front end, and a plurality of supports between the back end and the front end, the mount having a gap space between the back end and the front end;
   an elongate member having a blade portion and a carriage portion, the blade portion having a distal end, and a proximal end, the carriage portion forming a hook shape with the proximal end, the carriage portion positioned within the gap space, the carriage portion having a first aperture;
   a first lead screw in rotational engagement with the first aperture, the first lead screw having a proximal end and a distal end, the first lead screw being in mechanical engagement with the front end, the back end, and the carriage portion;
   wherein rotation of the first lead screw causes the elongate member to move along an axis of the first lead screw.
2. The blade assembly as previously described wherein the first lead screw is in mechanical engagement with a first blade gear at the proximal end of the first lead screw.
3. A blade assembly comprising:
   a mount comprising a back end, a front end, and a plurality of supports between the back end and the front end, the mount having a gap space between the back end and the front end;
   an elongate member having a body, a proximal end, a distal end and an end cap positioned substantially at the proximal end, the end cap being substantially orthogonal to the body of the elongate member, the end cap suspended over the front end of the mount;
   a carriage having a body and a bridge portion, the bridge portion positioned generally orthogonal to the carriage body, the bridge portion is mechanically engaged to the end cap, the bridge portion is suspended over the front end of the mount, the body being movably positioned within the gap space, the body further comprising a first aperture and a second aperture;
   a first lead screw in rotational engagement with the first aperture, the first lead screw having a proximal end in mechanical engagement with a first blade gear, the proximal end supported by the back end of the mount, and a distal end in rotational engagement with the front end of the mount;
   a second lead screw in rotational engagement with a second aperture, the second lead screw having a proximal end in mechanical engagement with a second blade gear, the proximal end supported by the back end of the mount, and a distal end in rotational engagement with the front end of the mount;
   a pinion gear in rotational engagement with the first and second blade gear;
   wherein rotation of one of the first or second lead screws cause the carriage to move within the gap space.
4. The blade assembly of any described herein, wherein the elongate member has an atraumatic profile.
5. The blade assembly of any described herein, wherein the elongate member further comprises an inward facing concave surface.
6. The blade assembly of any described herein, wherein the carriage is driven by the first blade gear.
7. The blade assembly of any described herein, wherein the carriage is driven by the second blade gear.
8. The blade assembly of any described herein, wherein the carriage is driven by both the first and second blade gears.
9. The blade assembly of any described herein, wherein the blade assembly is made from any combination of stainless steel, aluminum, polymers, ceramics, composites and metal alloys.
10. A tissue dilating apparatus for use with a robotic surgery system, the apparatus comprising:
   a frame having an interior surface and an exterior surface, the interior surface defining an aperture;
   a plurality of blade assemblies, wherein each blade assembly is mounted to the exterior surface of the frame, the elongate members being disposed in a substantially orthogonal orientation to the aperture, the plurality of elongate members defining a working channel and an axis of operation;
   a plurality of translation gears in movable mechanical engagement with the plurality of blade gears such that rotation of the translation gear causes the movement of the carriage in the gap space;
   wherein the translation gear moves in response to a motor to move the elongate members to alter the shape of the working channel.
11. The tissue dilating apparatus of any described herein, wherein the plurality of elongate members are not connected to each other.
12. The tissue dilating apparatus of any described herein, wherein the plurality of elongate members further comprises:
   a first elongate member;
   a second elongate member:
   wherein the first and second elongate members are moveably attached to the frame, the first and second elongate members having a proximal end in close proximity to the frame, and a distal end; and
   an expandable barrier mechanically engaged to the distal end of the first and second elongate members.
13. The tissue dilating apparatus of any described herein, wherein one or more of the plurality of elongate members further comprises:
   a carriage, the carriage bridging a distance of the frame, and the carriage being in mechanical engagement with one of a motor gear of the plurality of motor gears.
14. The tissue dilating apparatus of any described herein, wherein each elongate member is movable about a fulcrum.
15. The tissue dilating apparatus of any described herein, wherein the fulcrum is positioned in the aperture.
16. The tissue dilating apparatus of any described herein, wherein each of the plurality of the elongate members are independently movable.
17. The tissue dilating apparatus of any described herein, wherein the frame is rotatable about the axis of operation.
18. The tissue dilating apparatus of any described herein, wherein the apparatus further comprises a plurality of motors in mechanical engagement with the plurality of motor gears.
19. The tissue dilating apparatus of any described herein, wherein the apparatus further comprises an electronic controller for controlling the plurality of motors.
20. The tissue dilating apparatus of any described herein, wherein a force sensor is operable with at least one of the elongate members.
21. The tissue dilating apparatus of any described herein, further comprises:
   at least one neuromonitoring sensor in mechanical engagement with at least one of the elongate members.
22. The tissue dilating apparatus of any described herein, wherein the frame defines a plane that is substantially orthogonal to the axis of operation.
23. The tissue dilating apparatus of any described herein, wherein the set of elongate members are operable as a tissue retractor.
24. A robotic surgery system with a tissue dilating device, the system comprising:
   a robotic arm;
   a tissue dilating apparatus positioned at a distal end of the robotic arm, the apparatus comprising:
   a frame having an interior surface and an exterior surface, the interior surface defining an aperture;
   a plurality of elongate members, the plurality of elongate members being disposed in a substantially orthogonal orientation to the aperture, the plurality of elongate members defining a working channel and an axis of operation;
   a plurality of gears in movable mechanical engagement with the plurality of elongate members to move the elongate members in at least one dimension, the plurality of gears being mechanically engaged to the exterior surface;
   wherein the plurality of gears move in response to a plurality of motors to move the plurality of elongate members to alter the shape of the working channel;
   a computer with a user interface, the computer in electrical communication with the robotic arm and the tissue dilating apparatus; and
   an electronic controller directing the movement of the robotic arm and the tissue dilating apparatus, wherein the electronic controller is operated through the computer’s user interface.
25. A method of controlling a tissue dilating apparatus, the method comprising:
   providing, via at least one processor, a force actuation instruction to a motor;
   measuring, via an encoder, a movement of the motor to move a blade;
   determining, via a force sensor, a force resistance value on the blade in response to the movement of the motor; and
   compensating, via the at least one processor, for the force resistance value to move the blade to a predetermined position.
26. The method as previously described, wherein the force actuation instruction has a maximum force limit.

### Additional Aspects

1. A blade assembly comprising:
a carriage housing, the carriage housing having a front plate and a back plate, and a plurality of support members between the front plate and the back plate;
a first screw mechanically engaged to the carriage housing, the first screw passing through the back plate and coupled to the front plate, the first screw having a front end, a back end, and a first screw longitudinal axis;
a carriage mechanically engaged to the first screw, such that a rotation of the first screw is configured to displace the carriage along the first screw longitudinal axis of the first screw;
a blade mechanically engaged to the carriage, the blade positioned outside the carriage housing;
wherein the rotation of the first screw is configured to cause the carriage to move within the carriage housing, and further configured to cause a displacement of the blade outside of the carriage housing.

2. The blade assembly of any described herein, wherein the first screw comprises a first translation gear, the blade assembly further comprising:
a second screw substantially parallel to the first screw, the second screw being mechanically supported by the front plate and the back plate, the second screw having a second translation gear in substantially orthogonal alignment with the first translation gear of the first screw, the second screw having a second screw longitudinal axis; and
a pinion gear in rotational engagement with the first translation gear and the second translation gear, such that the pinion gear is configured to provide force to drive the second translation gear when the first translation gear is rotated;
wherein a rotation of the first and second translation gears is configured to move the carriage along the first screw longitudinal axis and the second screw longitudinal axis.

3. The blade assembly of any described herein, further comprising a second drive screw in parallel arrangement to the first screw, the second drive screw being rotated by a motor separate from the first screw.

4. The blade assembly of any described herein, further comprising a transverse screw mechanically engaged to the carriage housing and the carriage, the transverse screw being substantially orthogonal to the first screw.

5. The blade assembly of any described herein, where in the blade is made of at least one selected from a group consisting of a stainless steel, an aluminum, a titanium and a metal alloy.

6. The blade assembly of any described herein, further comprising a bridge portion configured to connect the blade and the carriage.

7. The blade assembly of any described herein, wherein the front plate has an aperture configured to receive at least one of the first screw or the second screw.

8. The blade assembly of any described herein, further comprising one or more sensors.

9. The blade assembly of any described herein, wherein the one or more sensors comprise at least one selected from a group consisting of a force sensor and a neurostimulator sensor.

10. The blade assembly of any described herein, wherein the first screw is directly coupled to the front plate.

11. A tissue dilating apparatus for use with a robotic surgical system, the apparatus comprising:
a frame having an interior and an exterior;
a plurality of blades disposed in the interior of the frame;
a plurality of blade assembly apparatus mounted circumferentially around the exterior of the frame, each blade assembly apparatus comprising:
a carriage housing,
a first screw mechanically engaged to the carriage housing,
a carriage mechanically engaged to the first screw, such that a rotation of the first screw configured to displace the carriage along a longitudinal axis length of the first screw,
one blade of the plurality of blades mechanically engaged to the carriage, the blade positioned outside of the carriage housing, wherein a rotation of the first screw is configured to cause the carriage to move within the carriage housing, and cause a displacement of the blade outside of the carriage housing and in the interior of the frame; and
a plurality of mechanical actuators engaged to the plurality of blade assembly apparatus.

12. The tissue dilating apparatus of any described herein, wherein the plurality of mechanical actuators are configured to move the plurality of blade assembly apparatus to create a working channel for a surgical instrument.

13. The tissue dilating apparatus of any described herein, wherein each of the plurality of mechanical actuators is coupled to a respective one of the plurality of blade assembly apparatus.

14. The tissue dilating apparatus of any described herein, wherein the plurality of blades form a tube.

15. The tissue dilating apparatus of any described herein, wherein the plurality of blades are individually moveable.

16. The tissue dilating apparatus of any described herein, further comprising:

a plurality of motors in removable mechanical engagement to the plurality of mechanical actuators.

17. The tissue dilating apparatus of any described herein, wherein each blade of the plurality of blades is connected to a corresponding carriage by a bridge, wherein the bridge extends outside the carriage housing and the frame.

18. The tissue dilating apparatus of any described herein, wherein the plurality of blade assembly apparatus are made of at least one selected from a group consisting of stainless steel, aluminum, and medical grade metal alloys.

19. The tissue dilating apparatus of any described herein, wherein the plurality of blades are configured to form a barrier.

20. The tissue dilating apparatus of any described herein, further comprising:

a helical braid mesh woven around at least a portion of the plurality of blades, the helical braid mesh configured to expand and contract with the plurality of blades and form a substantially contiguous barrier between the plurality of blades.

21. The tissue dilating apparatus of any described herein, wherein a respective distal end of each blade of the plurality of blades is configured to form the barrier.

22. The tissue dilating apparatus of any described herein, wherein the plurality of blades are individually movable in pairs.

23. The tissue dilating apparatus of any described herein, wherein a first pair of blades are movable such that proximal ends of the first pair are positioned closer together when distal ends of the first pair are spaced farther apart.

24. The tissue dilating apparatus of any described herein, wherein a second pair of blades are movable such that proximal ends of the second pair are spaced farther apart when distal ends of the second pair are positioned closer together.

25. The tissue dilating apparatus of any described herein, wherein the plurality of blades are configured to retain a substantially parallel configuration with respect to each other when moving.

26. A method of controlling a tissue dilating apparatus having a plurality of independently operable blades, the method comprising:
providing, to a controller, a reference command;
determining, by the controller, a position and an amount of motor torque relay to a motor, wherein the position is determined based at least in part upon the reference command;
transmitting a command to the motor to create the amount of motor torque to move a blade to the position,
receiving, from a sensor, sensor signal indicating a resistance force on the blade; and
determining, by the controller, a compensation motion based at least in part on the senor signal.

27. The method of any described herein, further comprising moving, by the controller, the plurality of independently operable blades to create a working channel for a surgical instrument.

28. The method of any described herein, wherein the plurality of independently operable blades form a tube.

29. The method of any described herein, wherein the sensor is a force sensor configured to measure the resistance force applied by tissues to the blades as the working channel is created.

30. The method of any described herein, wherein the compensation motion is determined continuously and in real-time.

Embodiments of the subject matter and the operations described in this specification may be implemented in digital electronic circuitry, or in computer software, firmware, or hardware, including the structures disclosed in this specification and their structural equivalents, or in combinations of one or more of them. Embodiments of the subject matter described in this specification may be implemented as one or more computer programs, i.e., one or more modules of computer program instructions, encoded on one or more computer storage medium for execution by, or to control the operation of, data processing apparatus, such as a processing circuit. A controller or processing circuit such as CPU may comprise any digital and/or analog circuit components configured to perform the functions described herein, such as a microprocessor, microcontroller, application-specific integrated circuit, programmable logic, etc. Alternatively, or in addition, the program instructions may be encoded on an artificially generated propagated signal, e.g., a machine-generated electrical, optical, or electromagnetic signal, that is generated to encode information for transmission to suitable receiver apparatus for execution by a data processing apparatus.

A computer storage medium may be, or be included in, a computer-readable storage device, a computer-readable storage substrate, a random or serial access memory array or device, or a combination of one or more of them. Moreover, while a computer storage medium is not a propagated signal, a computer storage medium may be a source or destination of computer program instructions encoded in an artificially generated propagated signal. The computer storage medium may also be, or be included in, one or more separate components or media (e.g., multiple CDs, disks, or other storage devices). Accordingly, the computer storage medium is both tangible and non-transitory.

The operations described in this specification may be implemented as operations performed by a data processing apparatus on data stored on one or more computer-readable storage devices or received from other sources. The term “data processing apparatus” or “computing device” encompasses all kinds of apparatus, devices, and machines for processing data, including by way of example a programmable processor, a computer, a system on a chip, or multiple ones, or combinations, of the foregoing The apparatus may include special purpose logic circuitry, e.g., an FPGA (field programmable gate array) or an ASIC (application specific integrated circuit). The apparatus may also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment may realize various different computing model infrastructures, such as web services, distributed computing and grid computing infrastructures.

A computer program (also known as a program, software, software application, script, or code) may be written in any form of programming language, including compiled or interpreted languages, declarative or procedural languages, and it may be deployed in any form, including as a standalone program or as a module, component, subroutine, object, or other unit suitable for use in a computing environment. A computer program may, but need not, correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform actions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user, embodiments of the subject matter described in this specification may be implemented on a computer having a display device, e.g., a CRT (cathode ray tube) or LCD (liquid crystal display) monitor, OLED (organic light emitting diode) monitor or other form of display for displaying information to the user and a keyboard and/or a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input. In addition, a computer may interact with a user by sending documents to and receiving documents from a device that is used by the user; for example, by sending web pages to a web browser on a user’s client device in response to requests received from the web browser.

While this specification contains many specific embodiment details, these should not be construed as limitations on the scope of any embodiments or of what may be claimed, but rather as descriptions of features specific to particular embodiments. Certain features described in this specification in the context of separate embodiments can also be implemented in combination in a single embodiment. Conversely, various features described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated in a single software product or packaged into multiple software products.

References to “or” may be construed as inclusive so that any terms described using “or” may indicate any of a single, more than one, and all of the described terms.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain embodiments, multitasking and parallel processing may be advantageous.

Having described certain embodiments of the methods and systems, it will now become apparent to one of skill in the art that other embodiments incorporating the concepts may be used. It should be understood that the systems described above may provide multiple ones of any or each of those components and these components may be provided on either a standalone machine or, in some embodiments, on multiple machines in a distributed system. The systems and methods described above may be implemented as a method, apparatus or article of manufacture using programming and/or engineering techniques to produce software, firmware, hardware, or any combination thereof. In addition, the systems and methods described above may be provided as one or more computer-readable programs embodied on or in one or more articles of manufacture. The term “article of manufacture” as used herein is intended to encompass code or logic accessible from and embedded in one or more computer-readable devices, firmware, programmable logic, memory devices (e.g., EEPROMs, ROMs, PROMs, RAMs, SRAMs, etc.), hardware (e.g., integrated circuit chip, Field Programmable Gate Array (FPGA), Application Specific Integrated Circuit (ASIC), etc.), electronic devices, a computer readable non-volatile storage unit (e.g., CD-ROM, floppy disk, hard disk drive, etc.). The article of manufacture may be accessible from a file server providing access to the computer-readable programs via a network transmission line, wireless transmission media, signals propagating through space, radio waves, infrared signals, etc. The article of manufacture may be a flash memory card or a magnetic tape. The article of manufacture includes hardware logic as well as software or programmable code embedded in a computer readable medium that is executed by a processor. In general, the computer-readable programs may be implemented in any programming language, such as LISP, PERL, C, C++, C#, PROLOG, or in any byte code language such as JAVA. The software programs may be stored on or in one or more articles of manufacture as object code.

The various descriptions and figures of the robotic dilator may be taken as generally informative and provide guidance on the use, manufacture and operation of the present disclosure. However, the description and figures should not be taken as limiting in any sense, as the description may be defined by the appended claims.

1. A blade assembly comprising:
a carriage housing, the carriage housing having a front plate and a back plate, and a plurality of support members between the front plate and the back plate;
a first screw mechanically engaged to the carriage housing, the first screw passing through the back plate and coupled to the front plate, the first screw having a front end, a back end, and a first screw longitudinal axis;
a carriage mechanically engaged to the first screw, such that a rotation of the first screw is configured to displace the carriage along the first screw longitudinal axis of the first screw;
a blade mechanically engaged to the carriage, the blade positioned outside the carriage housing;
wherein the rotation of the first screw is configured to cause the carriage to move within the carriage housing, and further configured to cause a displacement of the blade outside of the carriage housing.

2. The blade assembly of claim 1, wherein the first screw comprises a first translation gear, the blade assembly further comprising:
a second screw substantially parallel to the first screw, the second screw being mechanically supported by the front plate and the back plate, the second screw having a second translation gear in substantially orthogonal alignment with the first translation gear of the first screw, the second screw having a second screw longitudinal axis; and
a pinion gear in rotational engagement with the first translation gear and the second translation gear, such that the pinion gear is configured to provide force to drive the second translation gear when the first translation gear is rotated;
wherein a rotation of the first and second translation gears is configured to move the carriage along the first screw longitudinal axis and the second screw longitudinal axis.

3. The blade assembly of claim 1, further comprising a second drive screw in parallel arrangement to the first screw, the second drive screw being rotated by a motor separate from the first screw.

4. The blade assembly of claim 1, further comprising a transverse screw mechanically engaged to the carriage housing and the carriage, the transverse screw being substantially orthogonal to the first screw.

5. The blade assembly of claim 1, where in the blade is made of at least one selected from a group consisting of a stainless steel, an aluminum, a titanium and a metal alloy.

6. The blade assembly of claim 1, further comprising a bridge portion configured to connect the blade and the carriage.

7. The blade assembly of claim 1, wherein the front plate has an aperture configured to receive at least one of the first screw or the second screw.

8. The blade assembly of claim 1, further comprising one or more sensors.

9. The blade assembly of claim 8, wherein the one or more sensors comprise at least one selected from a group consisting of a force sensor and a neurostimulator sensor.

10. The blade assembly of claim 1, wherein the first screw is directly coupled to the front plate.

11. A tissue dilating apparatus for use with a robotic surgical system, the apparatus comprising:
a frame having an interior and an exterior;
a plurality of blades disposed in the interior of the frame;
a plurality of blade assembly apparatus mounted circumferentially around the exterior of the frame, each blade assembly apparatus comprising:
a carriage housing,
a first screw and a second screw mechanically engaged to the carriage housing,
a carriage mechanically engaged to the first screw and the second screw, such that a rotation of the first screw is configured to displace the carriage along a first longitudinal axis length of the first screw and a rotation of the second screw is configured to displace the carriage along a second longitudinal axis length of the second screw, and
one blade of the plurality of blades mechanically engaged to the carriage, the blade positioned outside of the carriage housing, wherein each of the first screw and the second screw is individually rotatable to cause the carriage to move within the carriage housing, and cause a displacement of the blade outside of the carriage housing and in the interior of the frame, wherein the first screw is configured to control the displacement of the blade at a first location along the blade, and the second screw is configured to control the displacement of the blade at a second location along the blade that is different from the first location; and
a plurality of mechanical actuators engaged to the plurality of blade assembly apparatus.

12. The tissue dilating apparatus of claim 11, wherein the plurality of mechanical actuators are configured to move the plurality of blade assembly apparatus to create a working channel for a surgical instrument.

13. The tissue dilating apparatus of claim 11, wherein each of the plurality of mechanical actuators is coupled to a respective one of the plurality of blade assembly apparatus.

14. The tissue dilating apparatus of claim 11, wherein the plurality of blades form a tube.

15. The tissue dilating apparatus of claim 11, wherein the plurality of blades are individually moveable.

16. The tissue dilating apparatus of claim 11, further comprising:
a plurality of motors in removable mechanical engagement to the plurality of mechanical actuators.

17. The tissue dilating apparatus of claim 11, wherein each blade of the plurality of blades is connected to a corresponding carriage by a bridge, wherein the bridge extends outside the carriage housing and the frame.

18. The tissue dilating apparatus of claim 11, wherein the plurality of blade assembly apparatus are made of at least one selected from a group consisting of stainless steel, aluminum, and medical grade metal alloys.

19. The tissue dilating apparatus of claim 11, wherein the plurality of blades are configured to form a barrier.

20. The tissue dilating apparatus of claim 11, further comprising:
a helical braid mesh woven around at least a portion of the plurality of blades, the helical braid mesh configured to expand and contract with the plurality of blades and form a substantially contiguous barrier between the plurality of blades.

21. The tissue dilating apparatus of claim 19, wherein a respective distal end of each blade of the plurality of blades is configured to form the barrier.

22. The tissue dilating apparatus of claim 11, wherein the plurality of blades are individually movable in pairs.

23. The tissue dilating apparatus of claim 22, wherein a first pair of blades are movable such that proximal ends of the first pair are positioned closer together when distal ends of the first pair are spaced farther apart.

24. The tissue dilating apparatus of claim 23, wherein a second pair of blades are movable such that proximal ends of the second pair are spaced farther apart when distal ends of the second pair are positioned closer together.

25. The tissue dilating apparatus of claim 11, wherein the plurality of blades are configured to retain a substantially parallel configuration with respect to each other when moving.

26. A method of controlling a tissue dilating apparatus having a plurality of independently operable blades, the method comprising:
providing, to a controller, a reference command;
determining, by the controller, a position and an amount of motor torque relay to a motor, wherein the position is determined based at least in part upon the reference command;
transmitting a command to the motor to create the amount of motor torque to move a blade to the position,
receiving, from a sensor, sensor signal indicating a resistance force on the blade; and
determining, by the controller, a compensation motion based at least in part on the senor signal.

27. The method of claim 26, further comprising moving, by the controller, the plurality of independently operable blades to create a working channel for a surgical instrument.

28. The method of claim 27, wherein the plurality of independently operable blades form a tube.

29. The method of claim 27, wherein the sensor is a force sensor configured to measure the resistance force applied by tissues to the blades as the working channel is created.

30. The method of claim 26, wherein the compensation motion is determined continuously and in real-time.
